(19) United States
(12) Patent Application Publication
(10) Pub. No.: US 2019/0000673 A1
(43) Pub. Date: Jan. 3, 2019

(54) OCULAR IMPLANTS FOR REDUCTION OF INTRAOCULAR PRESSURE AND METHODS FOR IMPLANTING SAME

(71) Applicant: GLAUKOS CORPORATION, San Clemente, CA (US)

(72) Inventors: Todd Raymond Fjield, Irvine, CA (US); Douglas Daniel Crimaldi, San Marcos, CA (US); Charles Raymond Kalina, Irvine, CA (US); Harold A. Heitzmann, Irvine, CA (US)

(21) Appl. No.: 15/746,392

(22) PCT Filed: Jul. 22, 2016
(86) PCT No.: PCT/US2016/043752

§ 371 Date: Jan. 19, 2018

Related U.S. Application Data
(60) Provisional application No. 62/195,719, filed on Jul. 22, 2015.

(51) Int. Cl. A61F 9/007

(52) U.S. Cl. CPC A61F 9/00781; A61F 2210/0061; A61F 2250/0069; A61F 2250/0067; A61F 2210/0014

(57) ABSTRACT

Devices and methods for treating intraocular pressure are disclosed. The devices include shunts for draining aqueous humor from the anterior chamber to the uveoscleral outflow pathway, including the supraciliary space and the suprachoroidal space.

### CROSS-REFERENCE TO RELATED APPLICATIONS

This application is a U.S. National Phase of PCT/US2016/0043752, having an international filing date of Jul. 22, 2016, designating the United States, and titled “OCULAR IMPLANTS FOR REDUCTION OF INTRAOCULAR PRESSURE AND METHODS FOR IMPLANTING SAME,” which claims the benefit of U.S. Provisional Patent Application No. 62/195,719, filed Jul. 22, 2015, and titled “OCULAR IMPLANTS FOR REDUCTION OF INTRAOCULAR PRESSURE AND METHODS FOR IMPLANTING SAME”, the entirety of which is incorporated by reference herein.

### FIELD

Several embodiments of the invention disclosed herein are directed to implants designed for reducing intraocular pressure within the eye. Embodiments disclosed herein also relate to a treatment of glaucoma and/or other ocular disorders wherein aqueous humor is permitted to flow out of an anterior chamber of the eye through a surgically implanted pathway.

### BACKGROUND

A human eye is a specialized sensory organ capable of light reception and is able to receive visual images. Aqueous humor is a transparent liquid that fills at least the region between the cornea, at the front of the eye, and the lens. A trabecular meshwork, located in an anterior chamber angle, which is formed between the iris and the cornea, normally serves as a drainage channel for aqueous humor from the anterior chamber so as to maintain a balanced pressure within the anterior chamber of the eye.

Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is a major treatment goal in all glaucomas.

In glaucomas associated with an elevation in eye pressure (intraocular hypertension), a main source of resistance to outflow is typically in the trabecular meshwork. The tissue of the trabecular meshwork normally allows the aqueous humor (hereinafter also referred to as “aqueous”) to enter Schlemm's canal, which then empties into aqueous collector channels in the posterior wall of Schlemm's canal and then into aqueous veins, which form the episcleral venous system. Aqueous is continuously secreted by ciliary bodies around the lens, so there is a constant flow of aqueous from the ciliary body to the anterior chamber of the eye. Pressure within the eye is determined by a balance between the production of aqueous and its exit through the trabecular meshwork (major route) and uveoscleral outflow (minor route) pathways. The portion of the trabecular meshwork adjacent to Schlemm's canal (the juxtacanilicular meshwork) can cause most of the resistance to aqueous outflow.

While a majority of the aqueous leaves the eye through the trabecular meshwork and Schlemm's canal, it is believed that at least about 10 to about 20 percent of the aqueous in humans leaves through the uveoscleral pathway. The degree with which uveoscleral outflow contributes to the total outflow of the eye appears to be species dependent. As used herein, the term “uveoscleral outflow pathway” is to be given its ordinary and customary meaning to a person of ordinary skill in the art (and it is not to be limited to a special or customized meaning), and refers without limitation to the space or passageway whereby aqueous exits the eye by passing through the ciliary muscle bundles located at or near an angle of the anterior chamber and into the tissue planes between the choroid and the sclera, which extend posteriorly to the optic nerve. From these tissue planes, it is believed that the aqueous travels through the surrounding scleral tissue and drains via the scleral and conjunctival vessels, or is absorbed by the uveal blood vessels.

It is unclear from studies whether the degree of physiologic uveoscleral outflow is pressure-dependent or pressure-independent. As used herein, the term “supraciliary space” is to be given its ordinary and customary meaning to a person of ordinary skill in the art (and it is not to be limited to a special or customized meaning), and refers without limitation to the portion of the uveoscleral pathway through the ciliary muscle and between the ciliary body and the sclera, and the term “suprachoroidal space” is to be given its ordinary and customary meaning to a person of ordinary skill in the art (and it is not to be limited to a special or customized meaning), and refers without limitation to the portion of the uveoscleral pathway between the choroid and sclera.

Glaucoma is broadly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye.

Open-angle glaucoma is any glaucoma in which the exit of aqueous through the trabecular meshwork is diminished while the angle of the anterior chamber remains open. For most cases of open-angle glaucoma, the exact cause of diminished filtration is unknown. Primary open-angle glaucoma is the most common of the glaucomas, and is often asymptomatic in the early to moderately advanced stages of glaucoma. Patients may suffer substantial, irreversible vision loss prior to diagnosis and treatment.

Most current therapies for glaucoma are directed toward decreasing intraocular pressure. Medical therapy includes topical ophthalmic drops or oral medications that reduce the production of aqueous or increase the outflow of aqueous. However, drug therapies for glaucoma are sometimes associated with significant side effects. The most frequent and perhaps most serious drawback to drug therapy, especially the elderly, is patient compliance. Patients often forget to take their medication at the appropriate times or else administer eye drops improperly, resulting in under- or overdosing. Patient compliance is particularly problematic with therapeutic agents requiring dosing frequencies of three times a day or more, such as pilocarpine. Because the effects of glaucoma are irreversible, when patients dose improperly, allowing ocular concentrations to drop below appropriate therapeutic levels, further permanent damage to vision occurs. Furthermore, current drug therapies are targeted to be deposited directly into the ciliary body where the aqueous is produced. And current therapies do not provide for a continuous slow-release of the drug. When drug therapy fails, surgical therapy is pursued.

Surgical therapy as currently pursued suffers from many disadvantages. These include a proper protocol for the medical practitioner to follow so as to introduce a suprachoroidal implant on a patient's eye without. Additionally, such a surgical procedure can involve many pre- and post-operative procedures which can add to overall inefficiency and cause the operation to involve a significant amount of costs. As such, these difficulties may make it harder for a patient to undertake such a surgery and reluctance on the part of the doctor to prescribe same.

### SUMMARY

As such, a need exists for a more facile, convenient, less invasive, and less traumatic means of delivering an intraocular pressure controlling implant into an eye while providing a cost-effective but safe surgical procedure. It is one advantage of certain embodiments of the invention(s) disclosed herein to provide delivery devices, systems and methods are provided for inserting an implant into an eye. The delivery or inserter devices or systems can be used to dispose or implant an ocular stent or implant, such as a shunt, in communication with the suprachoroidal space, uveal scleral outflow pathway, uveoscleral outflow path or supraciliary space of the eye. The implant can drain fluid from an anterior chamber of the eye to a physiologic outflow path of the eye, such as, the suprachoroidal space, uveal scleral outflow pathway, uveoscleral outflow path or supraciliary space. The delivery or inserter devices or systems can be used in conjunction with other ocular surgery, for example, but not limited to, cataract surgery through a preformed corneal incision, or independently with the inserter configured to make a corneal incision. The implant can be preloaded with or within the inserter to advantageously provide an operator friendly package, such as a sterile package, for use by the surgeon, doctor or operator.

As used herein, “implants” refers to ocular implants which can be implanted into any number of locations in the eye. In some embodiments, the ocular implants are drainage implants designed to facilitate or provide for the drainage of aqueous humor from the anterior chamber of an eye into a physiologic outflow pathway in order to reduce intraocular pressure. In some embodiments, the implant can be configured to provide a fluid flow path for draining aqueous humor from the anterior chamber to an uveoscleral outflow pathway. In some embodiments, the aqueous humor is diverted to the supraciliary space or the suprachoroidal space of the uveoscleral outflow pathway.

The term “implant” as used herein is a broad term, and is to be given its ordinary and customary meaning to a person of ordinary skill in the art (and it is not to be limited to a special or customized meaning), and refers without limitation to drainage shunts, stents, sensors, fluids, or any other device or substance capable of being permanently or temporarily inserted within an eye and left within a body after removal of a delivery instrument.

If desired, more than one implant of the same or different type may be implanted. For example, the implants disclosed herein may be used in combination with trabecular bypass shunts, such as those disclosed in U.S. Patent Publication 2004/0050392, and those described in U.S. Patent Publication 2005/0271704, filed Mar. 18, 2005. Additionally, implantation may be performed in combination with other surgical procedures, such as cataract surgery. All or a portion of the implant may be coated, e.g. with heparin, preferably in the flow path, to reduce blood thrombosis or tissue restenosis. Implants, in several embodiments are implanted into the eye using, for example instruments such as those disclosed in U.S. patent application Ser. No. 14/204276, filed Mar. 11, 2014 and International Patent Application No. PCT/US2014/024899, filed Mar. 12, 2014, each of which are incorporated herein by reference, in their entireties.

What is needed is an extended, site-specific treatment method for placing a drainage implant (preferably by an ab interno implantation procedure) for diverting aqueous humor in an eye from the anterior chamber to a location within the eye that will permit further reduction of intraocular pressure. One such location disclosed herein is the uveoscleral outflow pathway, which comprises the supraciliary space and the suprachoroidal space. In some embodiments of the present disclosure, a method is provided for implanting a drainage implant ab interno in an eye to divert aqueous humor from the anterior chamber to the supraciliary space.

There are provided, in accordance with the present disclosure, various embodiments of systems devices and methods for reducing intraocular pressure in an eye of a mammal. In several embodiments, there is provided a system for reducing intraocular pressure in an eye of a mammal, comprising: an elongate outer shell with proximal and distal ends and being shaped to define at least one fluid flow passageway and an expandable material configured to be passed through the implant and positioned within the physiological outflow space. In several embodiments, there is provided a system for reducing intraocular pressure in an eye of a mammal, comprising: an elongate outer shell with proximal and distal ends and being shaped to define at least one fluid flow passageway and an expandable material configured to be passed alongside or around the implant and positioned within the physiological outflow space. In some embodiments, the outer shell is dimensioned to have the proximal end be in fluid communication with an anterior chamber of an eye while the distal end is in fluid communication with a physiological outflow space of the eye and the fluid flow passageway comprises an inflow portion and an outflow portion. In several embodiments the expandable material is configured to expand in at least one dimension and create a void between the distal-most end of the implant and the tissues making up the physiological outflow space.

In several embodiments, the fluid flow passageway comprises an inflow portion positioned at the proximal end of the implant and an outflow portion positioned at the distal end of the implant. In several embodiments, the fluid flow passageway comprises one or more inflow portions positioned at or near the proximal end of the implant and at least one outflow portion positioned substantially along a distal portion of the elongate outer shell, with at least one outflow portion being at the distal end of the implant. Depending on the embodiment, the implant may be dimensioned to have the proximal end positioned within the anterior chamber of the eye and/or with the distal end within the supraciliary space or the suprachoroidal space. In several embodiments, the implant has a length of between about 15 and 25 mm.

In several embodiments, the expandable material allows ocular fluid to flow around at least a portion of the material in its expanded shape. In additional embodiments, the expandable material is at least partially permeable to ocular fluid and allows ocular fluid to flow through at least a portion of the material in its expanded shape. In still additional embodiments, the expandable material is porous and allows ocular fluid to flow through at least a portion of the material in its expanded shape. Combinations of expanding materials of differing porosity or permeability may be used in several embodiments. In several embodiments, the expandable material comprises a hydrogel, a porous foam, hyaluronic acid, a swellable polymer, or combinations thereof.

In several embodiments, the expandable material expands upon deployment from the implant into the physiological outflow space. Depending on the embodiment, expandable material may expand upon application of an external stimulus, or may be self-expanding.

In several embodiments, the expandable material is bioerodible. In some such embodiments, the expandable material bioerodes within about 2 weeks to 2 years.

In several embodiments the expandable material is encapsulated in a bioerodible material. In some such embodiments, the material encapsulating the expandable material bioerodes within about 3 seconds to about 30 minutes.

In several embodiments, the expandable material comprises at least two components maintained separately until the components are deployed from the implant into the physiological outflow space. In some embodiments, the expandable material comprises a material with shear-thinning characteristics. In some embodiments, the expandable material is positioned in the implant prior to introducing the implant through the anterior chamber. In additional embodiments, the expandable material is passed through the implant after introducing the implant through the anterior chamber.

In several embodiments, the implant comprises a bioerodible material. In several such embodiments, the implant bioerodes within about 2 weeks to 2 years.

In several embodiments, the systems additionally comprise at least one delivery instrument.

Also provided for in several embodiments, are methods for reducing intraocular pressure in an eye of a mammal. In several embodiments, the method comprises introducing an ocular implant through the anterior chamber of the eye, the ocular implant comprising an elongate outer shell with proximal and distal ends and being shaped to define at least one fluid flow passageway; advancing the implant into eye tissue such that the distal end is in fluid communication with a physiological outflow pathway for ocular fluid and the proximal end is in fluid communication with the anterior chamber of the eye and ejecting from the ocular implant and into the physiological outflow space an expandable material, wherein upon expansion, the expandable material expands in one or more dimensions to create a void in the physiological outflow space more posterior in the eye than the distal end of the implant, and wherein aqueous humor is conducted between the proximal and distal ends of the implant and into the physiological outflow space, thereby reducing intraocular pressure.

In several embodiments, the fluid flow passageway comprises an inflow portion positioned at the proximal end of the implant and an outflow portion positioned at the distal end of the implant. In several embodiments, the fluid flow passageway comprises one or more inflow portions positioned at or near the proximal end of the implant and at least one outflow portion positioned substantially along a distal portion of the elongate outer shell, with at least one outflow portion being at the distal end of the implant. In several embodiments, the proximal end is positioned within the anterior chamber of the eye, while in several embodiments the distal end is positioned within the physiological outflow space.

In some embodiments, the physiological outflow space is the supraciliary space or the suprachoroidal space. In several embodiments, the distal end is positioned within the supraciliary space or the suprachoroidal space.

In some embodiments the expandable material is positioned in the implant prior to introducing the implant through the anterior chamber while in some embodiments, the expandable material is passed through the implant after to introducing the implant through the anterior chamber.

In several embodiments, the expandable material is encapsulated in a bioerodible material. In several embodiments, the implant comprises a bioerodible material.

In accordance with some embodiments of the present invention, a method for reducing intraocular pressure in an eye of a mammal (e.g., human) is provided, comprising introducing an ocular implant into the anterior chamber of the eye, the ocular implant having proximal and distal ends, cutting eye tissue using a sharpened distal portion of the implant, advancing the implant from the anterior chamber into the cut eye tissue such that the distal end is located in the suprachoroidal space and the proximal end is located in the anterior chamber, and conducting aqueous humor between the proximal and distal ends of the implant.

An ocular implant is disclosed in accordance with some embodiments of the present invention. In some embodiments, the implant comprises a substantially straight, rigid, generally cylindrical body of a length no greater than 7 mm, preferably not greater than about 5 mm, and more preferably not greater than about 4 mm and not shorter than about 2 mm. In several embodiments, the body comprises a substantially flexible, generally cylindrical shell or body, that may be of length approximately 25 mm, including about 15 to about 18 mm, about 18 to about 21 mm, about 21 to about 23 mm, about 23 to about 25 mm, about 25 mm to about 27 mm, about 27 to about 30 mm, and overlapping ranges thereof. In several embodiments, the implant comprises at least one inner lumen that terminates at one or more outlets. The lumen is of a sufficient length to extend from an anterior chamber to a fluid outflow space of the eye (e.g., the suprachoroidal space). Depending the embodiment, one or more fluid flow regulating elements (e.g., valves, passageways, ducts, etc.) are provided for regulating fluid flow through the lumen.

A method for regulating intraocular pressure is disclosed in accordance with some embodiments of the present invention. In some embodiments, the method comprises placing an elongated implant in eye tissue with an inlet in an anterior chamber and an outlet in a uveoscleral outflow pathway of an eye, and utilizing intraocular pressure to apply a force to move a valve surface within the implant in a direction transverse to a longitudinal axis of the implant such that aqueous humor flows from the anterior chamber to the uveoscleral outflow pathway at intraocular pressures greater than a threshold pressure.

An intraocular implant is disclosed in accordance with some embodiments of the present invention. In some embodiments, the intraocular implant comprises one or more influent openings having a total cross-sectional flow area and communicating with an interior chamber within the implant, an outlet portion providing an egress flow path comprising one or more effluent openings, and a pressure regulation valve having a deflectable plate or diaphragm with a surface area exposed to fluid within the interior chamber, said surface area being substantially greater than the total cross-sectional flow area of the one or more influent openings. The valve is disposed between the interior chamber and the one or more effluent openings such that movement of the deflectable plate regulates flow from the interior chamber to the one or more effluent openings. The plate extends in a direction generally parallel to the inlet flow path and to the outlet flow path.

A method of performing surgery to lower intraocular pressure of an eye is disclosed in accordance with some embodiments of the present invention. In some embodiments, the method comprises providing an opening into an anterior chamber of the eye, inserting an instrument into the anterior chamber through said opening to perform a cataract extraction from the eye, providing an ocular implant having an inflow portion in fluid communication with an outflow portion, transporting the ocular implant from the opening through the anterior chamber of the eye to the anterior chamber angle of the eye, positioning the ocular implant such that the inflow portion of the ocular implant is positioned in the anterior chamber and the outflow portion of the ocular implant is positioned in the suprachoroidal space, and permitting aqueous humor to flow from the anterior chamber of the eye through the inflow portion of the ocular implant to the outflow portion of the ocular implant and into the suprachoroidal space of the eye.

A system for treating glaucoma in a patient is disclosed in accordance with some embodiments of the present invention. In some embodiments, the system comprises a drainage implant which, following implantation at an implantation site, drains fluid from the anterior chamber into a physiologic outflow space, such as the supraciliary space and a delivery instrument for implanting the drainage implant. The instrument has a distal end sufficiently sharp to penetrate eye tissue at an insertion site near the limbus of the patient's eye, and is sufficiently long to advance the implant transocularly from the insertion site across the anterior chamber to the implantation site. The instrument also has a sufficiently small cross section such that the insertion site self-seals without suturing upon withdrawal of the instrument from the eye. The instrument comprises a plurality of members longitudinally moveable relative to each other and a seal between the members to prevent aqueous humor from passing between the members proximal the seal when the instrument is in the eye.

A method for treating glaucoma is disclosed in accordance with some embodiments of the present invention. In some embodiments, the method comprises forming as incision in eye tissue located near the limbus of the eye, introducing a delivery instrument through the incision, the delivery instrument carrying a drainage device, implanting the drainage device in eye tissue near the scleral spur, without introducing a viscoelastic material into the anterior chamber, to establish a flow path for aqueous humor from the anterior chamber to a physiologic outflow path, and withdrawing the delivery instrument from the eye, wherein the incision is sufficient small that it is self-sealing once the delivery instrument is withdrawn.

A method for lowering intraocular pressure in a patient having at least one ocular shunt implanted in the trabecular meshwork to drain aqueous humor from the anterior chamber towards Schlemm's canal is disclosed in accordance with some embodiments of the present invention. In some embodiments, the method comprises introducing a drainage device through tissue adjacent the limbus into the anterior chamber, advancing the drainage device across the anterior chamber to a location near the scleral spur, and implanting the drainage device in eye tissue at a location spaced from the at least one ocular shunt and the trabecular meshwork to establish a flow path from the anterior chamber towards the suprachoroidal space.

A further aspect of the invention involves a system for treating glaucoma. The system comprises a plurality of implants, each having a distal end sufficiently sharp to extend through tissue into the suprachoroidal space, and an instrument having a chamber in which the implants are loaded for serial delivery into eye tissue.

Further aspects, features and advantages of the present invention will become apparent from the detailed description of the preferred embodiments of ocular implants, methods of implantation, and treatment courses that follow.

Certain embodiments of some of the inventions disclosed, taught or suggested herein are set forth in the appended claims.

For purposes of summarizing embodiments of the invention(s), certain aspects, advantages and novel features of the invention have been described herein above. Of course, it is to be understood that not necessarily all such advantages may be achieved in accordance with any particular embodiment of the invention. Thus, the invention may be embodied or carried out in a manner that achieves or optimizes one advantage or group of advantages as taught or suggested herein without necessarily achieving other advantages as may be taught or suggested herein.

All of these embodiments are intended to be within the scope of the invention herein disclosed. These and other embodiments of the invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

These and other features, aspects, and advantages of the present disclosure will now be described with reference to the drawings of embodiments, which embodiments are intended to illustrate and not to limit the disclosure.

FIG. 1 illustrates a schematic cross-sectional view of an eye with a delivery device being advanced across the anterior chamber.

FIG. 2 illustrates a schematic cross-sectional view of an eye with a delivery device being advanced adjacent the anterior chamber angle.

FIG. 3 illustrates a schematic cross-section view of an eye with a delivery device deploying an implant that extends between the anterior chamber and the uveoscleral outflow pathway.

FIGS. 4A-4G illustrate drainage implants in accordance with several embodiments disclosed herein.

FIG. 5 illustrates another drainage implant in accordance with embodiments disclosed herein.

FIG. 6 illustrates another drainage implant in accordance with embodiments disclosed herein.

FIG. 7 illustrates another drainage implant in accordance with embodiments disclosed herein including a core extending through a lumen of the implant.

FIG. 8 illustrates the implant of FIG. 7 with the core removed from the lumen of the implant.

FIG. 9 illustrates another drainage implant in accordance with embodiments disclosed herein including a ball-check pressure regulator.

FIG. 10 illustrates an exploded view of the implant of FIG. 9.

FIG. 11 illustrates another drainage implant in accordance with embodiments disclosed herein.

FIG. 12 illustrates an exploded view of the implant of FIG. 11.

FIG. 13 illustrates another drainage implant in accordance with embodiments disclosed herein.

FIG. 14 illustrates an exploded view of the implant of FIG. 13.

FIG. 15 illustrates a cross-sectional view of one embodiment of a deployment device with an implant extending therefrom.

FIG. 16 illustrates a perspective view of another embodiment of a deployment device.

FIG. 17 illustrates a schematic cross-sectional view of an eye with another delivery device being advanced across the anterior chamber.

FIG. 18 illustrates a schematic cross-sectional view of an eye with another delivery device being advanced across the anterior chamber.

FIG. 19 illustrates a cross-sectional view of another drainage implant in accordance with embodiments disclosed herein.

FIG. 20 illustrates a perspective view of another drainage implant in accordance with embodiments disclosed herein.

FIG. 21 illustrates a cross-sectional view of another embodiment of a deployment device.

FIG. 22 illustrates another deployment device in accordance with embodiments disclosed herein.

FIGS. 23A and 23B illustrate side views of the deployment device of FIG. 22.

FIG. 24 illustrates another deployment device in accordance with embodiments disclosed herein.

FIG. 25 illustrates a cross-sectional view of another drainage implant in accordance with embodiments disclosed herein.

FIGS. 26A-26C illustrate additional embodiments of a drainage implant in accordance with embodiments disclosed herein including a cap.

FIGS. 27A-27C illustrate additional embodiments of a drainage implant in accordance with embodiments disclosed herein including a flexible portion.

FIGS. 28A-28B illustrate embodiments of a reed-type valve in accordance with embodiments disclosed herein.

FIG. 29 illustrates another deployment device in accordance with embodiments disclosed herein.

FIG. 30 illustrates a cross-sectional view of another embodiment of a deployment device.

FIG. 31 illustrates a cross-sectional view of another embodiment of a deployment device.

FIG. 32 illustrates a cross-sectional view of another embodiment of a deployment device.

FIG. 33 illustrates a cross-sectional view of another embodiment of a deployment device.

FIGS. 34A and 34B are cross-sectional views of a shunt with sideports.

FIG. 35 is a cross-sectional view of another embodiment of a shunt with sideports.

FIG. 36 is a cross-sectional view of another embodiment of a shunt with sideports.

FIGS. 37A and 37B illustrate cross-sectional views of other drainage implants in accordance with embodiments disclosed herein.

FIG. 38 illustrates a cross-sectional view of another drainage implant in accordance with embodiments disclosed herein.

FIG. 39 illustrates a cross-sectional view of another drainage implant in accordance with embodiments disclosed herein.

FIGS. 40A and 40B illustrate cross-sectional views of other drainage implants in accordance with embodiments disclosed herein.

FIGS. 41A to 41H illustrate cross-sectional views of other drainage implants in accordance with embodiments disclosed herein.

FIGS. 42A to 42D illustrate cross-sectional views of other drainage implants in accordance with embodiments disclosed herein.

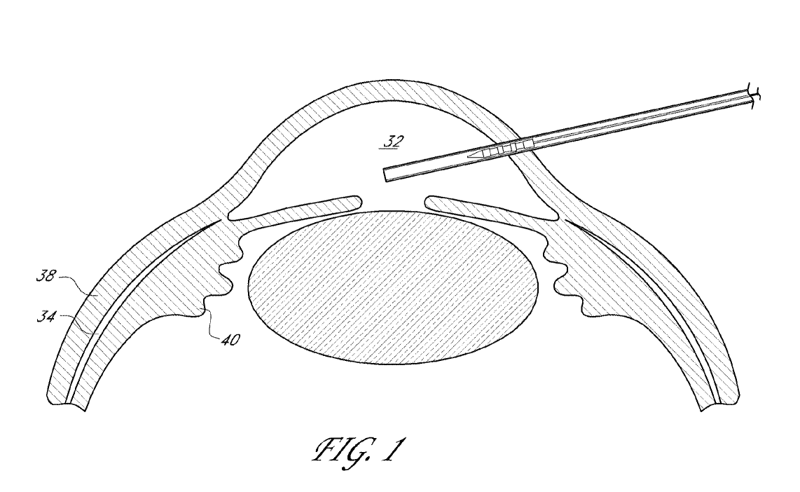

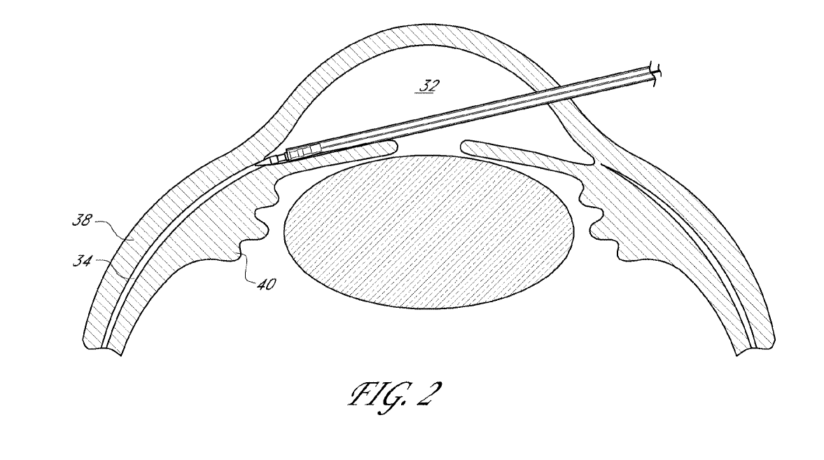

### DETAILED DESCRIPTION

The implants, systems and methods described herein are in connection with glaucoma treatment, and in particular with an extended, site-specific treatment method for placing a drainage shunt, or a shunt, for diverting aqueous humor in an eye from the anterior chamber to a location within the eye that will permit further reduction of intraocular pressure.

### General

Certain aspects of this disclosure relates to ophthalmic implant systems comprising a shunt which, following implantation at an implantation site, drains fluid from the anterior chamber into a physiologic outflow space, and a delivery instrument for implanting the shunt. One such outflow space disclosed herein is the uveoscleral outflow pathway, though additional outflow spaces are also contemplated.

In some embodiments, a shunt for providing a fluid flow path for draining aqueous humor from the anterior chamber of an eye to the uveoscleral outflow pathway to reduce intraocular pressure, is provided. In some embodiments, an instrument is provided for delivering and/or implanting a drainage shunt in an eye to divert aqueous humor from the anterior chamber to the uveoscleral outflow pathway. In some embodiments, a method is provided for implanting a drainage shunt in an eye to divert aqueous humor from the anterior chamber to the uveoscleral outflow pathway. In some embodiments, the aqueous humor is diverted to the superciliary space or the suprachoroidal space of the uveoscleral outflow pathway. In several embodiments, the implantation method is an ab interno method.

The term “shunt” as used herein is a broad term, and is to be given its ordinary and customary meaning to a person of ordinary skill in the art (and it is not to be limited to a special or customized meaning), and refers without limitation to an implant defining a fluid passage. The shunts may feature a variety of characteristics, described in more detail below, which facilitate the regulation of intraocular pressure. The mechanical aspects and material composition of the shunt are important for controlling the amount and direction of fluid flow. Therefore, various examples of shunt dimensions, features, tip configurations, material flexibility, coatings, and valve design, in accordance with some embodiments of the present disclosure, are discussed in detail below.

The delivery instruments, described in more detail below, may be used to facilitate delivery and/or implantation of the shunt to the desired location of the eye. The delivery instrument preferably is used to force the shunt into a desired position by application of a continual implantation force, by tapping the shunt into place using a distal portion of the delivery instrument, or by a combination of these methods. The design of the delivery instruments may take into account, for example, the angle of implantation and the location of the shunt relative to an incision. For example, in some embodiments, the delivery instrument may have a fixed geometry, be shape-set, or actuated. In some embodiments the delivery instrument may have adjunctive or ancillary functions. In some embodiments, the delivery instrument may be additionally be used to, for example, inject dye and/or viscoelastic fluid, to dissect, or as a guidewire.

The shunt can be advanced through the ciliary attachment tissue during deployment. This tissue typically is fibrous or porous, which is relatively easy to pierce or cut with a surgical device. The shunt can be advanced through this tissue and abut against the sclera once the shunt extends into the uveoscleral outflow pathway. The shunt can then slide within the uveoscleral outflow pathway along the interior wall of the sclera. As the shunt is advanced into the uveoscleral outflow pathway and against the sclera, the shunt will likely be oriented at an angle with respect to the interior wall of the sclera. The shunt is advanced until it reaches the desired implantation site within the uveoscleral outflow pathway. In some embodiments, the shunt is advanced into the ciliary body or ciliary muscle bundles to achieve drainage into the supraciliary space. In other embodiments, the shunt is advanced through the ciliary body or ciliary muscle bundles to achieve fluid communication between the anterior chamber and the suprachoroidal space. In still other embodiments, the shunt is advanced into the compact zone or through the compact to drain aqueous humor into the more distal portions of the suprachoroidal space.

### Shunts

The present disclosure relates to shunts (e.g., implant(s)) that provide a fluid flow path for draining aqueous humor from the anterior chamber of an eye to the uveoscleral outflow pathway to reduce intraocular pressure, preferably below episcleral venous pressure without hypotony.

The shunts can have an inflow portion and an outflow portion. In several embodiments, the shunts have at least one inflow portion and one or more outflow portions. In several embodiments, the number (and optionally surface area) of inflow portions are equivalent to the number (and optionally surface area) of the outflow portions. In additional embodiments, the number of inflow portions is not equivalent to the number of outflow portions. For example, in several embodiments, there is a plurality of inflow portions in order to provide redundancy in function, should one of the inflow portions become obstructed or cease to function. Likewise, in several embodiments, in several embodiments, a plurality of outflow portions is provided. In some embodiments, the plurality of outflow portions provide outflow to one or more physiological outflow spaces. In one embodiment, outflow portion of the shunt preferably is disposed at or near a distal end of the shunt. When the shunt is deployed, the inflow portion may be sized and configured to reside in the anterior chamber of the eye and the outflow portion may be sized and configured to reside in the suprachoroidal space. In some embodiments, the inflow portion is in fluidic communication with the anterior chamber, while not necessarily residing within the chamber. In some embodiments, the outflow portion may be sized and configured to reside in the supraciliary region of the uveoscleral outflow pathway, or the suprachoroidal space. In some embodiments, the outflow portion is in fluidic communication with one or more physiological outflow pathways, which may (or may not) require placement within the respective pathway.

At least one lumen can extend through the shunt. Preferably, there is at least one lumen that operates to conduct the fluid through the shunt. Each lumen preferably extends from an inflow end to an outflow end along a lumen axis. In some embodiments the lumen extends substantially through the longitudinal center of the shunt. In other embodiments, the lumen can be offset from the longitudinal center of the shunt.

A plurality of apertures can extend through the wall of the shunt, depending on the embodiment. In some embodiments, the apertures can extend through a middle portion of the shunt. In other embodiments the apertures can extend through other portions of the shunt. The plurality of apertures can provide several functions. One such function is that when the shunt is inserted into the suprachoroidal space, the apertures provide a plurality of routes through which the aqueous humor can drain. For example, once the shunt is inserted into the eye, if the shunt only has one outflow channel (e.g., one end of a lumen), that outflow channel can be plugged, for example, by the shunt's abutment against the interior wall of the sclera or the outer wall of the choroid. Additionally, the outflow channel can be clogged with tissue that is accumulated during the advancement of the shunt through the fibrous or porous tissue. The plurality of apertures provides a plurality of routes through which the fluid may flow to maintain patency and operability of the drainage shunt. In embodiments where the shunt has a porous body, the apertures can define surface discontinuities to assist in anchoring the shunt once deployed.

The shunt in some embodiments can include a distal edge that is sufficiently sharp to pierce eye tissue near the scleral spur of the eye, and that is disposed closer to the outlet portion than to the inlet portion. The distal edge can be sufficiently blunt so as not to substantially penetrate scleral tissue of the eye. In some embodiments, the shunts have a sharpened forward end and are self-trephinating, i.e., self-penetrating, so as to pass through tissue without pre-forming an incision, hole or aperture. The sharpened forward end can be, for example, conical or tapered. The tip can be sufficiently sharp to pierce eye tissue near the scleral spur of the eye. The tip also can be sufficiently blunt so as not to substantially penetrate scleral tissue of the eye. The taper angle of the sharpened end can be, for example, about 30°±15° in some embodiments. The radius of the tip can be about 70 to about 200 microns. In addition, the implant may have a substantially blunt or rounded distal end, so as to reduce tissue trauma during or after implantation, and the implant is positioned using a device having a distal portion configured to pierce ocular tissue.

In several embodiments, the body of the shunt includes at least one surface irregularity. The surface irregularity can comprise, for example, a ridge, groove, hole, depression, bump, or annular groove. The surface discontinuities or irregularities can also be formed by barbs or other projections, which extend from the outer surface of the shunt, to inhibit migration of the shunt from its deployed position. In some embodiments, the projections may comprise external ribbing to resist displacement of the shunt. The surface irregularity can interact with the tissue of the interior wall of the sclera. In some embodiments, the shunts are anchored by mechanical interlock between tissue and an irregular surface and/or by friction fit. In some embodiments, the shunt includes cylindrical recessed portions along an elongate body to provide enhanced gripping features during deployment and anchoring following implantation within the eye tissue. Depending on the embodiment, the surface irregularities are uniformly positioned along a long axis of the shunt. In additional embodiments, the surface irregularities are biased to a portion of the body of the implant (for example, in several embodiments, the irregularities are positioned more along a proximal portion of the implant).

The shunt may also incorporate fixation features, such as flexible radial extensions. The extensions may be separate pieces attached to the shunt, or may be formed by slitting the shunt wall, and thermally forming or mechanically deforming the extensions radially outward. If the extensions are separate pieces, they may be comprised of flexible material such as nitinol or polyimide. The extensions may be located at the anterior or posterior ends of the shunt, or both, to prevent extrusion of the shunt from its intended location. The flexibility of the fixation features will facilitate entry through the corneal incision, and also through the ciliary muscle attachment tissue.

In some embodiments, the body of the shunt has an outlet opening on a side surface to allow fluid flow. In some embodiments, the body of the shunt has a plurality of outlet openings on a side surface to allow fluid flow. In some embodiments, there is a plurality of outlet openings at one end of the shunt, such as the distal end. The openings can facilitate fluid flow through the shunt.

The shunt may have a cap, or tip, at one end. The cap can include a tissue-piercing end and one or more outlet openings. Each of the one or more outlet openings can communicate with at least one of the one or more lumens. In some embodiments cap can have a conically shaped tip with a plurality of outlet openings disposed proximal of the tip's distal end. In other embodiments, the cap can have a tapered angle tip. The tip can be sufficiently sharp to pierce eye tissue near the scleral spur of the eye. The tip also can be sufficiently blunt so as not to substantially penetrate scleral tissue of the eye. In some embodiments, the conically shaped tip facilitates delivery of the shunt to the desired location. In some embodiments, the cap has an outlet opening on a side surface to allow fluid flow. In some embodiments, the cap has a plurality of outlet openings on a side surface to allow fluid flow. In other embodiments, there is a plurality of outlet openings on the conical surface of the cap. The openings on the cap can facilitate fluid flow through the shunt. The opening may provide an alternate route for fluid flow which is beneficial in case the primary outflow portion of the shunt becomes blocked.

In some embodiments, multiple shunts are configured to be delivered during a single procedure. In some embodiments when multiple shunts are delivered, the shunts are arranged tandemly. The shunt can include a tip protector at one end. The tip protector can comprise a recess shaped to receive and protect, for example, the tip of an adjacent shunt. In some embodiments, the tip of the adjacent shunt has a conical shape. The recess may be shaped to contact the sides of the conical tip while protecting the more tapered tip, or end, from impact. The tip protector is particularly useful for delivery of multiple implants.

The shunts may be of varied lengths to optimize flows. In preferred embodiments, the length of the shunt is a length such that the outflow portion resides in the suprachoroidal space. In preferred embodiments, the length of the shunt is a length such that the outflow portion resides in the supraciliary space of the uveoscleral outflow pathway. In some embodiments, the length of the shunt is a length such that the outflow portion resides in the membranous region of the uveoscleral outflow pathway adjacent to the retina, while in other embodiments, the shunt has a length that extends distally past the membranous region. In some embodiments, the length of the shunt from the portion residing in the anterior chamber to the portion residing in the uveoscleral outflow pathway may be about 0.5 mm to about 5 mm. In preferred embodiments, the length of the shunt may be about 1.5 mm to about 5 mm. In more preferred embodiments, the length of the shunt may be about 2.0 mm. In some embodiments, the length of the shunt is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mm, and any value in between those listed. In additional embodiments, the body comprises a substantially flexible, generally cylindrical shell or body, that may be of length approximately 25 mm, including about 15 to about 18 mm, about 18 to about 21 mm, about 21 to about 23 mm, about 23 to about 25 mm, about 25 mm to about 27 mm, about 27 to about 30 mm, and overlapping ranges thereof.

The shunt can have an outer diameter that will permit the shunt to fit within a 23-gauge needle during deployment. The shunt can also have a diameter that is designed to be deployed with larger needles. For example, the shunt can also be deployed with 18-, 19- or 20-gauge needles. In other embodiments, smaller gauge applicators, such as a 23-gauge applicator, may be used. The shunt can have a constant diameter through most of the length of the shunt, or the shunt can have portions of reduced diameter, or cylindrical channels, e.g., annular grooves, between the inflow end and the outflow end. The distal end of the shunt can have a tapered portion, or a portion having a continually decreasing radial dimension with respect to the lumen axis along the length of the axis. The tapered portion preferably terminates with a smaller radial dimension at the outflow end. During deployment, the tapered portion can operate to form, dilate, and/or increase the size of, an incision or puncture created in the tissue. The tapered portion may have a diameter of about 23 gauge to about 30 gauge, and preferably about 25 gauge.

The diameter of one or more drainage lumens within the shunt may be varied to alter flow characteristics. The cross-sectional size of a shunt may be, for example, 0.1 mm to about 1.0 mm, or preferably about 0.3 mm to about 0.4 mm. A small cross-sectional size can be used to restrict flow. The cross-sectional shape of the shunt or a shunt may be any of a variety of cross-sectional shapes suitable for allowing fluid flow. For example, the cross-sectional shape of the shunt or shunt may be circular, oval, square, trapezoidal, rectangular, or any combination thereof.

In some embodiments, the shunt is configured to expand, either radially or axially, or both radially and axially. In some embodiments, the shunt may be self-expanding. In other embodiments, the shunt may be expanded by, for example, using a balloon device. In several embodiments, the expansion of the implant facilitates retention of the implant at the desired location within the eye.

The structure of the shunt may be flexible. At least a portion of the structure of the shunt may be flexible, or the whole structure may be flexible. In some embodiments, the structure of the shunt is accordion-like. The accordion-like structure provides flexibility. In other embodiments, at least a portion of the shunt is curved. In some embodiments, at least a portion of the shunt is straight. In some embodiments, the shunt has both curved and straight portions.

The shunt is preferably made of one or more biocompatible materials. Suitable biocompatible materials include polypropylene, polyimide, glass, nitinol, polyvinyl alcohol, polyvinyl pyrolidone, collagen, chemically treated collagepolyether sulfone, poly(styrene-isobutyl-styrene), Pebax, acrylic, polyolefin, polysilicon, polypropylene, hydroxyapetite, titanium, gold, silver, platinum, other metals, ceramics, plastics and a mixture thereof. The shunts can be manufactured by conventional sintering, micro machining, laser machining, and/or electrical discharge machining.

In some embodiments, the shunt is made of a flexible material. In other embodiments, the shunt is made of a rigid material. In some embodiments, a portion of the shunt is made from flexible material while another portion of the shunt is made from rigid material. The body can have an outer surface of which at least a portion is porous. Some embodiments include porosity that can be varied by masking a portion of the exterior with a band. Where the shunts include a porous body, the cross-section and porosity can be calibrated (down to 0.5 micrometers) to control the flow rates of aqueous humor through the shunt.

In some embodiments, at least a portion of the shunt is made of a material capable of shape memory. A material capable of shape memory may be compressed, and upon release may expand axially or radially, or both axially and radially, to assume a particular shape. In some embodiments, at least a portion of the shunt has a preformed shape. In other embodiments, at least a portion of the shunt is made of a superelastic material. In some embodiments, at least a portion of the shunt is made up nitinol. In other embodiments, at least a portion of the shunt is made of a deformable material.

The body of the shunt can comprise material that includes a drug and can include a coating. The coating can include a bioactive agent. The coatings can be, for example, a drug eluting coating, an antithrombogenic coating, and a lubricious coating. The bioactive agent can be selected from the group consisting of: heparin, TGF-beta, an intraocular pressure-lowering drug, and an anti-proliferative agent. Materials that may be used for a drug-eluting coating include parylene C, poly (butyl methacrylate), poly (methyl methacrylate), polyethylene-co-vinyl acetate, and other materials known in the art.

The shunt can further comprise a biodegradable material in or on the shunt. The biodegradable material can be selected from the group consisting of poly(lactic acid), polyethylene-vinyl acetate, poly(lactic-co-glycolic acid), poly(D,L-lactide), poly(D,L-lactide-co-trimethylene carbonate), collagen, heparinized collagen, poly(caprolactone), poly(glycolic acid), and a copolymer. All or a portion of the shunt may be coated, e.g. with heparin, preferably in the flow path, to reduce blood thrombosis or tissue restenosis.

The flow of fluid through the shunt can be configured to be regulated to a flow rate that will reduce the likelihood of hypotony in the eye. In some embodiments, the intraocular pressure is maintained at about 8 mm Hg. In other embodiments, the intraocular pressure is maintained at pressures less than about 8 mmHg, for example the intraocular pressure may be maintained between about 6 mm Hg and about 8 mm Hg. In other embodiments, the intraocular pressure is maintained at pressures greater than about 8 mm Hg. For example, the pressures may be maintained between about 8 mmHg and about 18 mm Hg, and more preferably between 8 mm Hg and 16 mm Hg. In some embodiments, the flow rate can be limited to about 2.5 μL/min or less. In some embodiments the flow rate can be limited to between about 1.9 μL/min and about 3.1 μL/min.

For example, the Hagen-Poiseuille equation suggests that a 4 mm long stent at a flow rate of 2.5 μL/min should have an inner diameter of 52 mm to create a pressure gradient of 5 mm Hg above the pressure in the suprachoroidal space.

The shunt may or may not comprise means for regulating fluid flow through the shunt. Means for regulating fluid flow can include flow restrictors, pressure regulators, or both. Alternatively, in some embodiments the shunt has neither a flow restrictor nor a pressure regulator. Regulating flow of aqueous humor can comprise varying between at least first and second operational states in which flow through of aqueous humor is more restricted in a first state and less restricted in a second state. Increasing the restriction to flow when changing from the second state to the first state can involve moving a valve toward a valve seat in a direction generally parallel or generally normal to a line connecting the proximal and distal ends of the shunt.

In some embodiments, the outflow portion of the shunt is sized and configured to reside in the supraciliary region of the uveoscleral outflow pathway. In embodiments where the outflow portion of the shunt is sized and configured to reside in the supraciliary region of the uveoscleral outflow pathway, there is a lesser need for means for regulating fluid flow through the shunt.

The means for flow restriction may be, for example, a valve, long length, small cross section, or any combination thereof. In some embodiments, the flow of fluid is restricted by the size of a lumen within the shunt, which produces a capillary effect that limits the fluid flow for given pressures. The capillary effect of the lumen allows the shunt to restrict flow and provides a valveless regulation of fluid flow.

The flow path length may be increased without increasing the overall length of the shunt by creating a lumen with a spiral flow path. A lumen within the shunt is configured to accommodate placement therein of a spiral flow channel core that is configured to provide preferred flow restriction. In effect, the spiral flow channel provides an extended path for the flow of fluid between the two ends of the shunt that is greater than a straight lumen extending between the ends of the shunt. The extended path provides a greater potential resistance of fluid flow through the shunt without increasing the length of the shunt. The core could have a single spiral flow channel, or a plurality of spiral flow channels for providing a plurality of flow paths through which fluid may flow through the shunt. For example, the core can have two or more spiral flow channels.

In some embodiments, the means for flow regulation comprises a pressure regulating valve. The valve can open when fluid pressure within the anterior chamber exceeds a preset level. Intraocular pressure may be used to apply a force to move a valve surface within the shunt in a direction transverse to a longitudinal axis of the shunt such that aqueous humor flows from the anterior chamber to the uveoscleral outflow pathway at intraocular pressures greater than a threshold pressure.

A shunt may have any number of valves to restrict flow and/or regulate pressure. The valve is preferably located between the interior chamber and one or more effluent openings such that movement of the deflectable plate regulates flow from the interior chamber to the one or more effluent openings. A variety of valves are useful with the shunt for restricting flow. In some embodiments, the valve is a unidirectional valve. The pressure relief valve can comprise a ball, a ball seat and a biasing member urging the ball towards the ball seat. In some embodiments, the valve is a reed-type valve. In a reed valve, for example, one end of the valve may be fixed to a portion of the shunt. The body of the reed valve is capable of being deflected in order to allow flow. Pressure from fluid in the anterior chamber can deflect the body of the reed valve, thereby causing the valve to open.

In some embodiments, the shunt includes a pressure regulation valve having a deflectable plate or diaphragm with a surface area exposed to fluid within the interior chamber, the surface area being substantially greater than the total cross-sectional flow area of the one or more influent openings. Such a valve can be disposed between the interior chamber of the shunt and the one or more effluent openings such that movement of the deflectable plate regulates flow from the interior chamber to the one or more effluent openings. The plate can extend in a direction generally parallel to the inlet flow path and to the outlet flow path.

The shunt can include ball-check pressure regulator. For example, when the intraocular pressure exceeds a particular pressure, the ball-check pressure regulator will open and permit fluid to flow between the anterior chamber and the uveoscleral outflow pathway. When the intraocular pressure reaches a second, lower pressure, the ball-check pressure regulator will close and limit or inhibit fluid from being conducted to the suprachoroidal space. The ball-check pressure regulator will remain closed until the intraocular pressure again reaches the particular pressure, and at which time the ball-check valve will reopen to permit or enhance drainage of fluid to the uveoscleral outflow pathway. Accordingly, the shunt provides drainage of the anterior chamber through the shunt based on the intraocular pressure levels and provides a means for reducing the likelihood for over-draining the anterior chamber and causing hypotony.

### Delivery Instruments

Another aspect of the systems and methods described herein relates to delivery instruments for implanting a shunt for draining fluid from the anterior chamber into a physiologic outflow space. In some embodiments, the shunt is inserted from a site transocularly situated from the implantation site. The delivery instrument can be sufficiently long to advance the shunt transocularly from the insertion site across the anterior chamber to the implantation site. At least a portion of the instrument can be flexible. The instrument can comprise a plurality of members longitudinally moveable relative to each other. In some embodiments, at least a portion of the delivery instrument is curved. In some embodiments, a portion of the delivery instrument is rigid and another portion of the instrument is flexible.

In some embodiments, the delivery instrument has a distal curvature. The distal curvature of the delivery instrument may be characterized as a radius of approximately 10 to 30 mm, and preferably about 20 mm.

In some embodiments, the delivery instrument has a distal angle. The distal angle may be characterized as approximately 90 to 180 degrees relative to the proximal segment 150 of the delivery instrument, and preferably about 145 degrees. The angle can incorporate a small radius of curvature at the “elbow” so as to make a smooth transition from the proximal segment of the delivery instrument to the distal segment. The length of the distal segment may be approximately 0.5 to 7 mm, and preferably about 2 to 3 mm.

In some embodiments, the instruments have a sharpened forward end and are self-trephinating, i.e., self-penetrating, so as to pass through tissue without pre-forming an incision, hole or aperture. Alternatively, a trocar, scalpel, or similar instrument can be used to pre-form an incision in the eye tissue before passing the shunt into such tissue.

For delivery of some embodiments of the ocular shunt, the instrument can have a sufficiently small cross section such that the insertion site self-seals without suturing upon withdrawal of the instrument from the eye. An outer diameter of the delivery instrument can be no greater than about 18 gauge and is not smaller than about 27 gauge.

For delivery of some embodiments of the ocular shunt, the incision in the corneal tissue is preferable made with a hollow needle through which the shunt is passed. The needle has a small diameter size (e.g., 18 or 19 or 20 or 21 or 22 or 23 or 24 or 25 or 26 or 27 gauge) so that the incision is self-sealing and the implantation occurs in a closed chamber with or without viscoelastic. A self-sealing incision also can be formed using a conventional “tunneling” procedure in which a spatula-shaped scalpel is used to create a generally inverted V-shaped incision through the cornea. In a preferred mode, the instrument used to form the incision through the cornea remains in place (that is, extends through the corneal incision) during the procedure and is not removed until after implantation. Such incision-forming instrument either can be used to shunt the ocular shunt or can cooperate with a delivery instrument to allow implantation through the same incision without withdrawing the incision-forming instrument. Of course, in other modes, various surgical instruments can be passed through one or more corneal incisions multiple times.

Once into the anterior chamber, a delivery instrument can be advanced from the insertion site transocularly into the anterior chamber angle and positioned at a location near the scleral spur. Using the scleral spur as a reference point, the delivery instrument can be advanced further in a generally posterior direction to drive the shunt into eye tissue at a location just inward of the scleral spur toward the iris. The placement and implantation of the shunt can be performed using a gonioscope or other conventional imaging equipment. The delivery instrument preferably is used to force the shunt into a desired position by application of a continual implantation force, by tapping the shunt into place using a distal portion of the delivery instrument, or by a combination of these methods. Once the shunt is in the desired position, it may be further seated by tapping using a distal portion of the delivery instrument.

The delivery instrument can include an open distal end with a lumen extending therethrough. Positioned within the lumen is preferably a pusher tube that is axially movable within the lumen. The pusher tube can be any device suitable for pushing or manipulating the shunt in relation to the delivery instrument, such as, for example, a screw, a rod, a stored energy device such as a spring. A wall of the delivery instrument preferably extends beyond pusher tube to accommodate placement within the lumen of a shunt. The shunt can be secured in position. For example, the shunt can be secured by viscoelastic or mechanical interlock with the pusher tube or wall. When the shunt is brought into position adjacent the tissue in the anterior chamber angle, the pusher tube is advanced axially toward the open distal end of the delivery instrument. As the pusher tube is advanced, the shunt is also advanced. When the shunt is advanced through the tissue and such that it is no longer in the lumen of the delivery instrument, the delivery instrument is retracted, leaving the shunt in the eye tissue.

Some embodiments include a spring-loaded pusher system. The spring-loaded pusher preferably includes a button operably connected to a hinged rod device. The rod of the hinged rod device engages a depression in the surface of the pusher, keeping the spring of the pusher in a compressed conformation. When the user pushes the button, the rod is disengaged from the depression, thereby allowing the spring to decompress, thereby advancing the pusher forward.

In some embodiments, an over-the wire system is used to deliver the shunt. The shunt can be delivered over a wire. Preferably, the wire is self-trephinating. The wire can function as a trocar. The wire can be superelastic, flexible, or relatively inflexible with respect to the shunt. The wire can be pre-formed to have a certain shape. The wire can be curved. The wire can have shape memory, or be elastic. In some embodiments, the wire is a pull wire. The wire can be a steerable catheter.

In some embodiments, the wire is positioned within a lumen in the shunt. The wire can be axially movable within the lumen. The lumen may or may not include valves or other flow regulatory devices.

In some embodiments, the delivery instrument is a trocar. The trocar may be angled or curved. In some embodiments, the trocar is flexible. In other embodiments the trocar is relatively rigid. In other embodiments, the trocar is stiff. In embodiments where the trocar is stiff, the shunt is preferably relatively flexible. The diameter of the trocar can be about 0.001 inches to about 0.01 inches. In some embodiments, the diameter of the trocar is 0.001, 0.002, 0.004, 0.005, 0.006, 0.007, 0.008, 0.009, or 0.01 inches.

In some embodiments, delivery of the shunt is achieved by applying a driving force at or near the distal end of the shunt. The driving force can be a pulling or a pushing applied to the end of the shunt.

The instrument can include a seal to prevent aqueous humor from passing through the delivery instrument and/or between the members of the instrument when the instrument is in the eye. The seal can also aid in preventing backflow. Suitable seals for preventing leakage include, for example, an o-ring, a coating, a hydrophilic agent, a hydrophobic agent, and combinations thereof. The coating can be, for example, a silicone coat such as MDX™ silicone fluid. In some embodiments, the instrument is coated with the coating and a hydrophilic or hydrophobic agent. In some embodiments, one region of the instrument is coated with the coating plus the hydrophilic agent, and another region of the instrument is coated with the coating plus the hydrophobic agent. The delivery instrument can additionally comprise a seal between various members comprising the instrument. The seal can comprise a hydrophobic or hydrophilic coating between slip-fit surfaces of the members of the instrument. The seal can be disposed proximate of the drainage shunt when carried by the delivery instrument. Preferably, the seal is present on at least a section of each of two devices that are machined to closely fit with one another.

The delivery instrument can include a distal end having a beveled shape. The delivery instrument can include a distal end having a spatula shape. The beveled or spatula shape can have a sharpened edge. The beveled or spatula shape can include a recess to contain the shunt. The recess can include a pusher or other suitable means to push out or eject the shunt.

The delivery instrument can be configured to deliver multiple shunts. In some embodiments, when multiple shunts are delivered, the shunts can be arranged in tandem.

### Procedures

For delivery of some embodiments of the ocular shunt, the implantation occurs in a closed chamber with or without viscoelastic.

The shunts may be placed using an applicator, such as a pusher, or they may be placed using a delivery instrument having energy stored in the instrument, such as disclosed in U.S. Patent Publication 2004/0050392, filed Aug. 28, 2002, which is attached hereto as Appendix A and the entirety of which is incorporated herein by reference and made a part of this specification and disclosure. In some embodiments, fluid may be infused through an application to create an elevated fluid pressure at the forward end of the shunt to ease implantation.

In some embodiments, the shunt is implanted through the fibrous attachment of the ciliary muscle to the sclera. This fibrous attachment zone extends about 0.5 mm posteriorly from the scleral spur, as shown between the two arrows (10) in FIG. 17.

In some embodiments it is desirable to deliver the shunt ab interno across the eye, through a small incision in the limbus. The overall geometry of the system makes it advantageous that the delivery instrument incorporates a distal curvature, or a distal angle. In the former case, the shunt can be flexible to facilitate delivery through the curvature. In the latter case, the shunt can be relatively rigid. The delivery instrument can incorporate a shunt advancement element that is flexible enough to pass through the distal angle.

In some embodiments, during clinical use, the shunt and delivery instrument can be advanced together through the limbus, across the iris, and through the ciliary muscle until the shunt tip is located in the uveoscleral outflow pathway. In other embodiments, the operator can then simultaneously push on a pusher device while pulling back on the delivery instrument, such that the shunt tip maintains its location in the uveoscleral outflow pathway. The shunt is released distally from the delivery instrument, and the delivery instrument is retracted proximally. At this point, the shunt can still ride on the distal end of the pusher. The pusher can then be withdrawn, leaving the shunt in place in the tissue. Finally, the delivery instrument can be withdrawn from the anterior chamber through the incision.

In some embodiments, a viscoelastic can be injected into the suprachoroidal space to create a chamber or pocket between the choroid and sclera which can be accessed by a shunt. Such a pocket could expose more of the choroidal and scleral tissue area, and increase uveoscleral outflow, causing a lower IOP. In some embodiments, the viscoelastic material can be injected with a 25 or 27G cannula, for example, through an incision in the ciliary muscle attachment.

In some embodiments, a hyperosmotic agent can be injected into the suprachoroidal space. Such an injection can delay IOP reduction. Thus, hypotony can be avoided in the acute postoperative period by temporarily reducing choroidal absorption. The hyperosmotic agent can be, for example glucose, albumin, HYPAQUE™ medium, glycerol, or poly(ethylene glycol). The hyperosmotic agent can breakdown or wash out as the patient heals, resulting in a stable, acceptably low IOP, and avoiding transient hypotony.

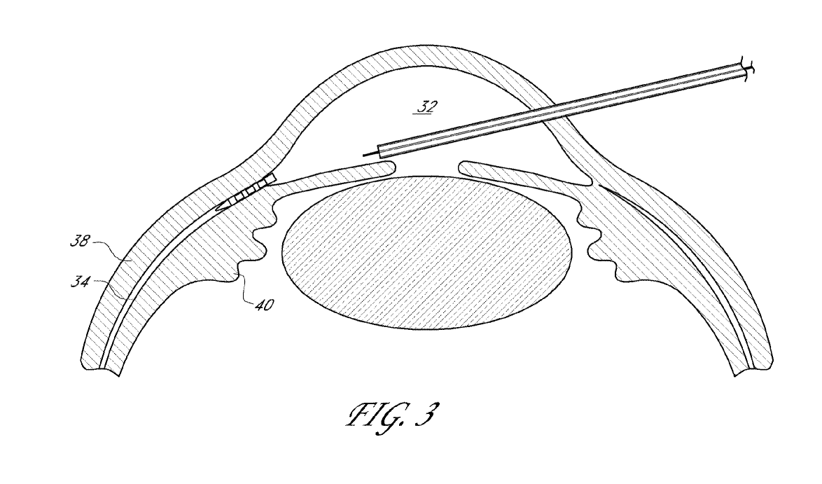

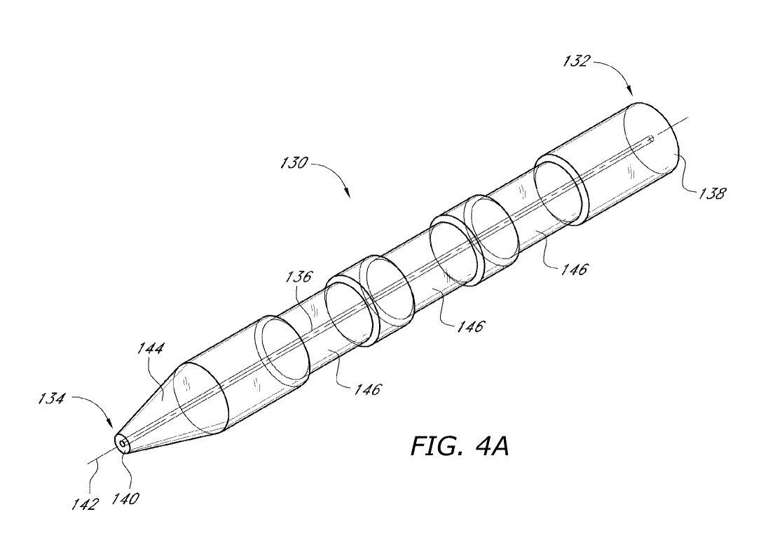

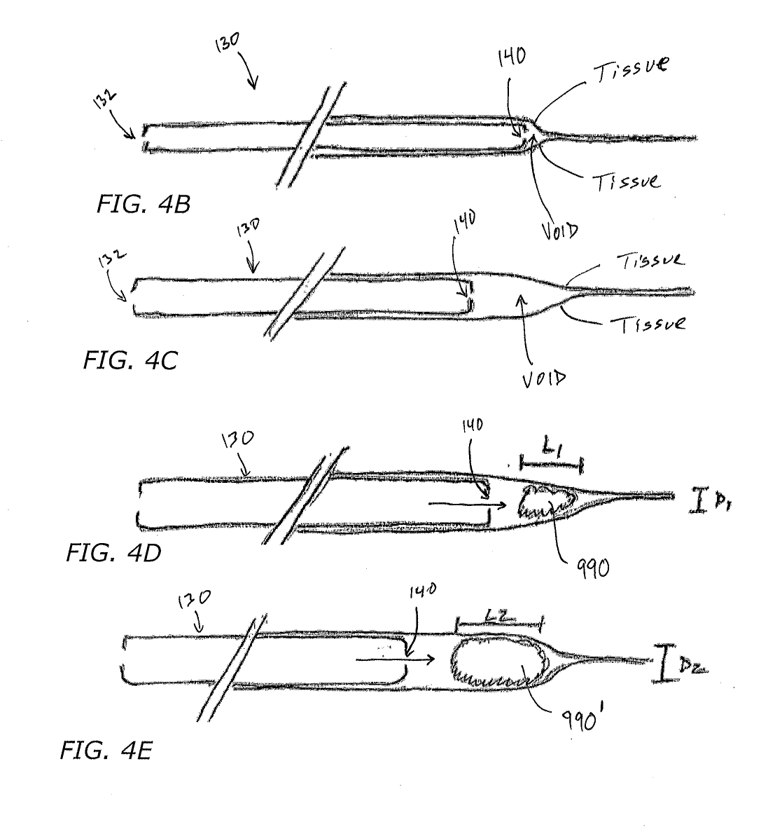

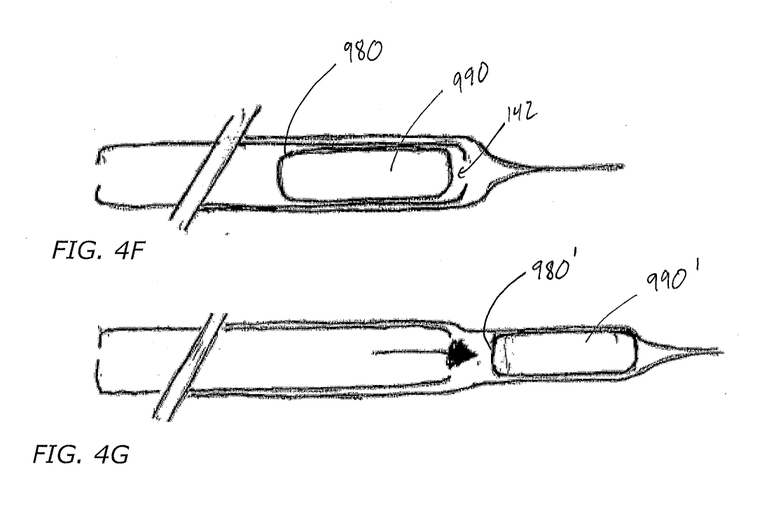

Embodiments Illustrated in FIGS. 4A-4G

FIG. 4A illustrates one embodiment of a shunt 130 that is operable to drain fluid from the anterior chamber to the suprachoroidal space. The shunt 130 has an inflow portion 132 and an outflow portion 134. When the shunt is deployed, the inflow portion 132 is sized and configured to reside in the anterior chamber of the eye and the outflow portion 134 is sized and configured to reside in the uveoscleral outflow pathway. Extending through the shunt 130 is preferably at least one lumen 136 that operates to conduct the fluid through the shunt 130. Each lumen 136 preferably extends from an inflow end 138 to an outflow end 140 along a lumen axis 142.

The shunt 130 preferably has an outer diameter that will permit the shunt 130 to fit within a 21-gauge or 23-gauge needle during deployment, however, larger or smaller gauge needles may also be used. The shunt 130 can also have a diameter that is designed to be deployed with larger needles. For example, the shunt 130 can also be deployed with 18-, 19- or 20-gauge needles. The shunt 130 can have a constant diameter through most of the length of the shunt 130, or the shunt 130 can have portions of reduced diameter, or cylindrical channels, e.g., annular grooves 146, between the inflow end 138 and the outflow end 140. The annular grooves 146 produce an irregular outer surface that can operate to mechanically lock or anchor the shunt 130 in place following deployment of the shunt 130. Of course, such surface discontinuities or irregularities can also be formed by barbs or other projections, which extend from the outer surface of the shunt, to inhibit migration of the shunt 130 from its deployed position.

The outflow portion 134 of the shunt 130 preferably is disposed at or near a distal end of the shunt 130. In the illustrated embodiment, the distal end has a tapered portion 144, or a portion having a continually decreasing radial dimension with respect to the lumen axis 142 along the length of the axis 142. The tapered portion 144 preferably terminates with a smaller radial dimension at the outflow end 140. During deployment, the tapered portion can operate to form, dilate, and/or increase the size of, an incision or puncture created in the tissue. For example, the outflow end 140 can operate as a trocar to puncture or create an incision in the tissue. The outflow end 140 of the shunt 130 can be advanced through the puncture or incision in the tissue created by the outflow end 140. Following advancement of the outflow end 140 of the shunt 130, the tapered portion 144 can be advanced through the puncture or incision. The tapered portion 144 will operate to stretch or expand the tissue around the puncture or incision to accommodate the increasing size of the tapered portion 144 as it is advanced through the tissue. When the stretched tissue passes over the cylindrical channels 146 having a reduced diameter, the stretched tissue will retract generally to fill the cylindrical channels 146 and will abut the edges of the shunt 130 having a greater diameter. The interaction of the tissue and the edges of the shunt 130 will provide an anchor for the shunt 130 following deployment to inhibit shunt migration.

The tapered portion 144 can also facilitate proper location of the shunt 130 into the supraciliary or suprachoroidal spaces. For example, the shunt 130 is preferably advanced through the tissue within the anterior chamber angle during deployment. This tissue typically is fibrous or porous, which is relatively easy to pierce or cut with a surgical device, such as the tip of the shunt 130. The shunt 130 can be advanced through this tissue and abut against the sclera once the shunt extends into the uveoscleral outflow pathway. As the shunt 130 abuts against the sclera, the tapered portion 144 preferably provides a generally rounded edge or surface that facilitates sliding of the shunt 130 within the suprachoroidal space along the interior wall of the sclera. For example, as the shunt 130 is advanced into the uveoscleral outflow pathway and against the sclera, the shunt 130 will likely be oriented at an angle with respect to the interior wall of the sclera. As the tip of the shunt 130 engages the sclera, the tip preferably has a radius that will permit the shunt 130 to slide along the sclera instead of piercing or substantially penetrating the sclera. As the shunt 130 slides along the sclera, the tapered portion 144 will provide an edge against which the shunt 130 can abut against the sclera and reduce the likelihood that the shunt will pierce the sclera.

Once the shunt 130 is deployed in position with the inflow portion 132 residing in the anterior chamber and the outflow portion 134 residing in the uveoscleral outflow pathway, aqueous humor flows from the anterior chamber to the uveoscleral outflow pathway through the lumen 136 of the shunt. The flow of fluid is preferably restricted by the size of the lumen 136, which produces a capillary effect that limits the fluid flow for given pressures. The capillary effect of the lumen allows the shunt to restrict flow and provides a valveless regulation of fluid flow. The flow of fluid through the shunt 130 is preferably configured to be restricted to flow rated that will reduce the likelihood of hypotony in the eye. For example, in some embodiments, the flow rate can be limited to about 2.5 μL/min or less. In some embodiments the flow rate can be limited to between about 1.9 μL/min and about 3.1 μL/min. In other applications, a plurality of shunts 130 can be used in a single eye to conduct fluid from the anterior chamber to the uveoscleral outflow pathway. In such applications, the cumulative flow rate through the shunts preferably is within the range of about 1.9 μL/min to about 3.1 μL/min, although the flow rate for each of the shunts can be significantly less than about 2.5 μL/min. For example, if an application called for implantation of five shunts, then each shunt 130 can be configured to have a flow rate of about 0.5 μL/min.

While the lumen is depicted in FIG. 4A as extending substantially through the longitudinal center of the shunt 130, in some embodiments, the lumen can be offset from the longitudinal center of the shunt. For example, while FIG. 4A depicts the shunt as having a tapered portion 144 that terminates substantially where the tapered portion 144 meets the lumen 136, the lumen 136 can be offset from the center of the shunt 130 such that lumen 136 opens along one of the sides of the tapered portion 144. Accordingly, the tapered portion 144 can terminate at a location offset from the lumen axis 142 and can extend beyond the point at which the interior lumen 136 and the exterior tapered portion 144 meet.

The shunt 130 preferably comprises any of the materials previously described above. The shunt 130 can be fabricated through conventional micro machining techniques or through procedures commonly used for fabricating optical fibers. For example, in some embodiments, the shunts 130 are drawn with a bore, or lumen, extending therethrough. In some embodiments, the tapered portion 144 at the outflow portion 134 can be constructed by shearing off an end tubular body. This can create a tapered portion 144 that can be used to puncture or incise the tissue during implantation and dilate the puncture or incision during advancement of the shunt 130. Other materials can be used for the shunt of FIG. 4, and other methods of manufacturing the shunt 130 can also be used. For example, the shunt 130 can be constructed of metals or plastics, and the shunts can be machined with a bore that is drilled.

The shunt 130 of FIG. 4A represents a shunt having a simple construction that provides a simple implantation procedure. The construction of the shunt provides for the opportunity to vary the size of the shunt 130 or the lumen 136. Additionally, the shunt 130 is able to be constructed in small needles. For example, the shunt 130 can fit within a needle for the implantation procedure. The needle preferably has a size of about 18 gauge to about 23 gauge, and most preferably about 23 gauge. The shunt also need not have a unitary configuration; that is, be formed of the same piece of material. For example, a proximal portion of the shunt can be formed of glass drawn to have at least one small diameter lumen. A distal portion of the shunt can be a cap formed of a different material. The cap includes a tissue-piercing end and one or more outlet openings. Each of the one or more outlet openings communicates with at least one of the one or more lumens in the proximal portion. In one preferred mode, the cap has a conically shaped tip with a plurality of outlet openings disposed proximal of the tip's distal end.

The shunt is preferably made of one or more biocompatible materials. Suitable biocompatible materials include polyvinyl alcohol, polyvinyl pyrolidone, collagen, chemically treated collagen, polytetrafluoroethylene, expanded polytetrafluoroetylene, fluorinate polymer, fluorinate elastomer, flexible fused silica, silica, silicone, polyurethane, poly(methyl methacrylate), poly (butyl methacrylate), acrylic, polyolefin, polysilicon, polypropylene, hydroxyapetite, titanium, gold, silver, platinum, and a mixture thereof

Additional embodiments are described below and schematically illustrated in FIGS. 4B-4G. While several embodiments of the implants disclosed herein are configured to be positioned within a physiological outflow space, once positioned, the surrounding tissue may return, at least in part, to its original position preimplantation. As a result, an opening, void or other space (see, e.g., FIG. 4B/4C) that was formed as a result of positioning the implant within the desired outflow space, may be formed, but can be reduced over time or could further improve outflow if the void were larger volume. If the void is reduced over time, or smaller than optimal (keeping in mind the desire not to traumatize the ocular tissue or induce inflammation), the effective outflow-promoting nature of the implant may be lessened.

In several embodiments, an implant according to several embodiments disclosed herein is positioned in, for example, the suprachoroidal space. Beyond the distal most portion of the implant, a void is formed because the shape of the implant has caused a separation between the choroid and the sclera. Still more posterior to the void, the tissue layers are undisturbed and juxtaposed with one another. Such a situation is schematically illustrated in FIG. 4B, with the void just beyond the outflow portion 140 of the implant 130 shown as a small space that is formed before the two layers of tissue into which the implant was positioned remain in or come back into juxtaposition with one another. While fluid outflow may still be improved vis-à-vis outflow prior to implantation of the implant, in several embodiments maintenance or generation of a larger void further enhances the therapeutic efficacy of the implant. The latter is illustrated schematically in FIG. 4C, where the void between the outflow portion 140 of the implant 130 has a greater volume as compared to the void in FIG. 4B. The larger volume void schematically illustrated in FIG. 4C provides additional outflow space for removal of ocular fluid, increased exposed surface areas of the choroid and sclera for enhanced outflow from the suprachoroidal space, and a coordinate reduction in intraocular pressure.

In view of the above, in several embodiments, it is desirable to maintain an additional gap or void around the implant outlets in the physiological outflow space. Depending on the embodiment, the additional gap or void may be positioned beyond the distal-most portion of the implant, surrounding the implant along the long axis of the implant, or combinations thereof. Thus, by way of example only, implantation of an implant according to several embodiments disclosed herein (optionally having a blunt distal end) results in a void between the distal-most end of the implant in the ocular tissue in which the implant is positioned. By generating a “volume effect”, for example, drainage is facilitated in several embodiments.

Depending on the embodiment, an increased void can be accomplished in several different fashions. In several embodiments, a material is deployed from the distal portion of the implant to a position in the physiological outflow space that is more posterior than the distal portion of implant. This is schematically illustrated, for example, in FIG. 4D. in several embodiments, a material 990 is deployed through a distally positioned outflow region 140 of the implant 130. In several embodiments, the material 990 is an expandable material. As shown schematically in FIG. 4D, upon deployment into the physiological outflow space, the material 990 may have a first dimension (D1) and a first length (L1). Upon expansion, the material 990 adopts an expanded shape with a second dimension (D2) and a second length (L2). Depending on the embodiment, the degree of expansion (e.g., D1 to D2 and/or L1 to L2) can be an increase of at least about 10%, about 15%, about 20%, about 25%, about 30%, about 40%, about 50%, about 75%, about 100%, or more (and any degree of expansion between those listed). Additionally, depending on the embodiment the expanding material need not expand uniformly. In other words in one embodiment, the length of the expanding material may expand to a greater degree than the diameter of the expanding material. Alternatively, the diameter may expand to a greater degree than the length. Depending on the embodiment the expansion is optionally tailored to an individual. For example a first subject may benefit from an implant that delivers an expandable material that expands greater with respect to its diameter as compared to its length, while a second subject may benefit more, based on their ocular anatomy, from an implant that expands to a greater degree in terms of diameter as compared to length. In some embodiments the expansion of the material is amorphous and functions simply to assist in creating an additional void beyond the distal-most portion of the implant.

In additional embodiments, the expanding material need not be delivered through the implant, in some embodiments the expandable material can be delivered around the implant. For example, in some embodiments, a delivery device that is configured to deliver the implant can comprise a conduit for delivery of the expanding material alongside the implant. In some embodiments, delivery of the expanding material separately from the implants is advantageous because the amount and/or position of the expandable material can be separately controlled. For example, it may be advantageous to deliver the expanding material to a final position that is alongside the implant (e.g., positioned between the outer shell of the implants and the surrounding ocular tissue). Delivery of the expanding material to such a position can be in addition to, or in place of, delivery of the expanding material to a position more posterior to the distal end of the implant. In some embodiments, delivery of the expanding material can be concurrent with, or after positioning the implant. In still additional embodiments, delivery of the expanding material can be prior to insertion of the implant, such as via a cleft into the supraciliary/suprachoroidal space. Additionally, delivery of the expanding material can be achieved using a specialized inserter, as discussed above, or in additional embodiments through the use of a separate conduit specifically for delivery of the expanding material.

Depending on the embodiment, the amount of expanding material that is delivered can be varied. In some embodiments, the volume of expanding material ranges from about 5 μL to about 200 μL, including about 5 μL to about 10 μL, about 10 μL to about 15 μL, about 15 μL to about 20 μL, about 20 μL to about 25 μL, about 25 μL to about 30 μL, about 30 μL to about 35 μL, about 35 μL to about 40 μL, about 40 μL to about 45 μL, about 45 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 125 μL, about 125 μL to about 150 μL, about 150 μL to about 175 μL, about 175 μL about 200 μL, and any amount in between those ranges listed, including endpoints. Depending on the embodiment, the total volume of the expanding material that is delivered can be delivered in a single delivery (e.g. injection), two separate deliveries, or a plurality of separate deliveries. Depending on the embodiment, when separate deliveries are used, the deliveries need not be performed at the same time. For example, in some embodiments, a first delivery of expanding material can be performed prior to insertion of the implant, while a second delivery can be performed concurrent with, or after, delivery of the implant.

In several embodiments, the material is a porous material that allows ocular fluid to pass through the material. Depending on the embodiment the relative porosity of the material can be tailored to achieve a desired outflow rate of ocular fluid. In other words, the material can be used to further fine-tune the increased outflow that is achieved using the implant itself. In some embodiments therefore the implant may be a standardized implant while the material ejected from the implant is used to tailor the physiological outflow rate specifically for an individual patient. In other embodiments both the material and the implant are personalized to a particular subject, synergistically acting with one another to provide an optimal therapeutic effect.

Use of a porous material is preferred in some embodiments. In additional embodiments, a material that does not have specific pores (e.g., no direct apertures, but rather a sponge-like “tortuous pathways”) but is still permeable or semi-permeable to ocular fluid can be used. In effect, use of porous and/or otherwise at least partially permeable materials prevents the material deployed from acting as a plug and rather allows the material to act in a more stent-like fashion (e.g., maintaining flow through the physiological outflow pathway while still structurally supporting the tissues of the pathway in an open configuration).

Non-limiting embodiments of expandable materials include, but are not limited to, viscoelastic materials, hydrogels, swellable hydrophilic polymers (e.g., swellable hydrophilic aliphatic polyurethane), other forms of polymers (e.g., porous polyurethane foams), hydrogel particles, for example, polyacrylamide, cross-linked polymers, poly2-hydroxyethylmethacrylate (HEMA) polyethylene oxide, polyAMPS and polyvinylpyrrolidone, or naturally derived hydrogels such as agarose, methylcellulose, hyaluronan or others, or combinations of the above. Hydrogel is a three-dimensional network of cross-linked, hydrophilic polymer chains. The hydrophilicity of the polymer chains cause the hydrogel to swell in the presence of sufficient quantities of fluid. In other embodiments, the expandable material is foam, collagen, or any other similar biocompatible material that swells, solidifies or gels, or otherwise expands. In some embodiments, the expandable material begins to expand immediately on contact with an appropriate solvent (e.g., ocular fluid). In other embodiments, expansion occurs after passage of a short period of time, such that the implant can be fully positioned in the desired target site prior to expansion of the material. Preferred solvents that induce expansion include water, saline, ocular fluid, aqueous humor, or other biocompatible solvents. In certain embodiments, the expandable material comprises a material that exhibits shear thinning characteristics, e.g., when exposed to shear forces (such as being injected or extruded through the distal portion of the implant) the material is pliable or liquid-like. Upon relief from shear forces, such as when the material is fully ejected from the distal portion of the implant into the physiological outflow pathway, the expandable material exhibits more rigid characteristics (though, depending on the embodiment some degree of pliability is desirable).

In several embodiments, the expanding material expands at a rate that is predetermined to provide sufficient expansive force without causing tissue trauma. Different rates may be used, for example depending on the age or current medical status of the subject. In some embodiments, the expanding material expands to its full dimension within about 3 to 5 seconds, within about 5 to 10 seconds, within about 10 to 20 seconds, within about 20 to 30seconds, within about 30 to 60 seconds, or within about 90 seconds (or any times in between those listed. In additional embodiments, a slower rate of expansion is desired. In such embodiments, the expanding material expands to its full dimensions within about 3 to 5 minutes, within about 5 to 8 minutes, within about 8 to 12 minutes, within about 12 to 15 minutes, within about 15 to 20 minutes, within about 20 to 30 minutes, or longer (or within any time in between those listed).

In several embodiments, the expandable material 900 is encapsulated or otherwise coated within one or more biodegradable materials (see e.g., 980 in FIG. 4F). With this approach, after expulsion of the material from the implant (990′ in FIG. 4G) the encapsulating material 980′ begins to bioerode. In some embodiments, the encapsulating material 980′ is configured to bioerode within a few days of implantation of the implant in the physiological outflow space and expulsion of the material 990′ from the distal portion of the implant. As described above, in several embodiments the implant and/or the material can be tailored to the needs of a specific patient. Building on this, the encapsulating material 980′, depending on the embodiment can be configured to erode at a greater or lesser rate, depending on the needs of a given particular patient. In some embodiments, the encapsulating material 980′ begins to bioerode on the order of minutes to hours after expulsion from the implant (e.g., about two minutes, about five minutes, about 10 minutes, about 30 minutes, about 60 minute, about two hours, about four hours, about six hours, about 12 hours, or about 24 hours, or any time in between those listed above). In additional embodiments, the encapsulating material 980′ is configured to begin to bioerode within about one to three days, within about 2 to 4 days, within about 4 to 6 days, within about 6 to 10 days, within about 10 to 14 days, within about 14 to 21 days, within about 21 to 30 days, within about 30 to 60 days, or within about 60 to 90 days (or any timeframe in between those listed). As also discussed herein, in several embodiments the implant itself comprises a biodegradable material. Depending on the embodiment the bioerodible material used to construct the implant may or may not be the same as the bioerodible encapsulating material. In several embodiments, the bioerodible material used to construct the implant is configured to bioerode on a longer timeframe than that of the encapsulating material, e.g., on the order of several months to several years. As a result, depending on the embodiment, after a certain period of time not only will expanding material discharge from an implant bioerode, but the implant itself will also bioerode. While the embodiment of FIGS. 4F-4G illustrate the encapsulating material enveloping the expandable material 900 in bulk, the encapsulating material can surround each of a plurality of micro-spheres of the expandable material. The micro-spheres can exhibit flowable characteristics so as to be expelled out the distal end of the implant. Once expelled, the encapsulating material erodes allowing the expandable material to expand. Alternatively, the expandable material can expand causing the encapsulating material to fracture, thereby allowing for continued expansion.

The material deployed form the implant may expand as a result of, for example, exposure to the ocular fluid within the physiological outflow space. The material may also expand as a result of being exposed to a physiological temperature. Additional external stimuli that can induce expansion of the material include, but are not limited to, light energy, electromagnetic energy, heat, ultrasound, radio frequency, or laser energy. Similarly, one or more of the above listed stimuli may also be used to trigger the bioerosion of the encapsulating material 980′.

In some embodiments, the expanding material comprises one or more components that react with one another. In such embodiments, the material components are maintained as separate (e.g., in the lumen of the implant) and expelled from the implant (e.g., injected into the outflow space) which allows the components to mix and react, thereby causing expansion upon expulsion from the distal end of the implant. In some embodiments, co-expulsion from the implant allows for micro static mixing of the one or more components that make up the expanding material, thereby allowing reaction between the various components and expansion within the physiological outflow space.

Depending on the embodiment, the expandable material can be positioned within a lumen of the implant prior to surgically positioning the implant within the ocular tissue of the subject. In essence, a preloaded implant/expanding material system is provided. In some embodiments, however, the implant is positioned within the desired location of a subject ocular tissue and subsequently (preferably during the same surgical procedure) the expanding material is loaded into the implant and expelled from the distal end of the implant into the desired space to create and/or to maintain a void of the desired dimensions and volume. Depending on the embodiment, and the expanding material used in that particular embodiment, a plunger-like device can be used to, for example, to expel an expanding material 990 comprising an encapsulating material 980. Plunger-like devices can also be used for those types of expanding material that are not encapsulated. Similarly, air pressure, liquid pressure, energy, or vibration can be used to expel the expanding material from an implant. In additional embodiments the expanding material can be delivered to its ultimate position by passing a cannula or other injection device and directly delivering the expanding material to its final position within the physiological outflow space (analogous to the implant forming a sheath through which the cannula is passed).

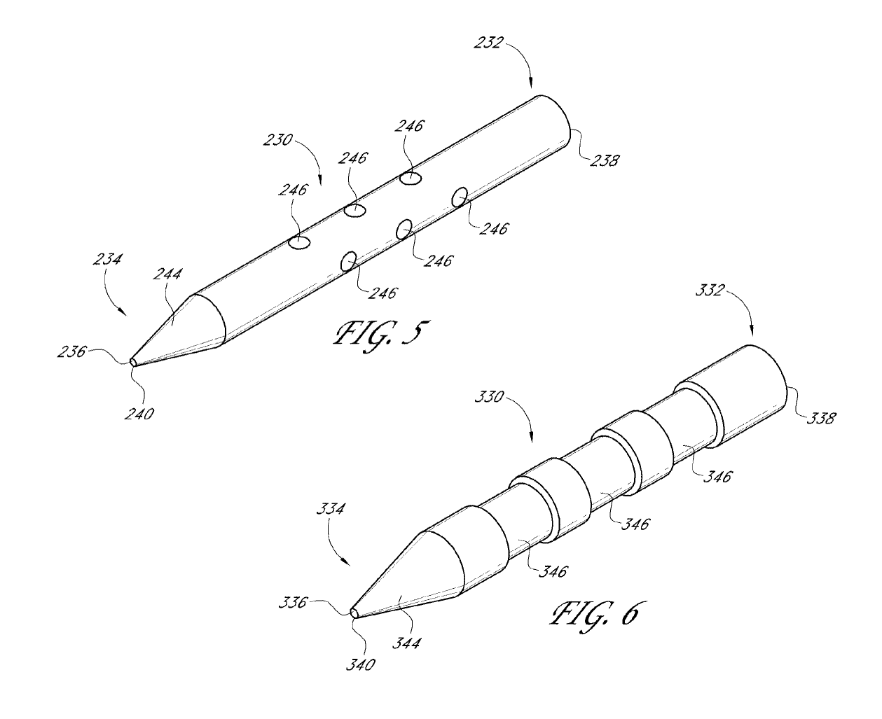

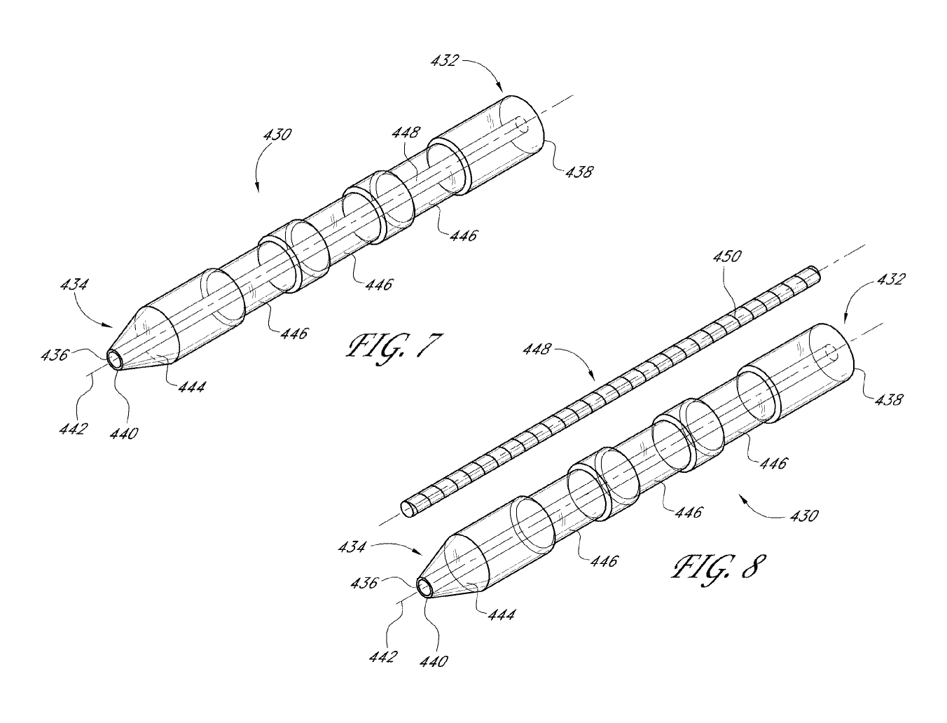

Embodiments Illustrated in FIGS. 5 and 6

Additional embodiments of shunts are depicted in FIGS. 5. FIG. 5 illustrates a shunt having a relatively similar construction as that of FIG. 4. FIG. 5 illustrates an embodiment of a shunt 230 having an elongate body with an inflow portion 232 and an outflow portion 234. A lumen(s) 236 preferably extends between an inflow end 238 and an outflow end 240. Proximate the outflow end 240 is preferably a tapered portion 244 having a construction similar to the embodiments described above with respect to FIG. 4. Alternatively, the bodies of the shunts can be formed of a porous material which has one or more flow paths from the inflow portion 232 to the outflow portion 240.

FIG. 5 depicts a plurality of apertures 246 extending through the wall of the shunt 230. While the apertures 246 are depicted as extending through a middle portion of the shunt 230, the apertures can extend through other portions of the shunt 230. For example, the apertures 246 can also extend through the outflow portion 234, or more particularly, through the tapered portion 244. The plurality of apertures 246 can provide several functions. One such function is that when the shunt 230 is inserted into the uveoscleral outflow pathway, the apertures 246 provide a plurality of routes through which the aqueous humor can drain. For example, once the shunt 230 is inserted into the eye, if the shunt 230 only has one outflow channel (e.g., one end of a lumen), that outflow channel can be plugged, for example, by the shunt's abutment against the interior wall of the sclera or the outer wall of the choroid. Additionally, the outflow channel can be clogged with tissue that is accumulated during the advancement of the shunt 230 through the fibrous or porous tissue. The plurality of apertures 246 provides a plurality of routes through which the fluid may flow to maintain patency and operability of the drainage shunt 230. In embodiments where the shunt has a porous body, the apertures 246 can define surface discontinuities to assist in anchoring the shunt once deployed.

FIG. 6 depicts embodiments of a shunt 330 having an elongate body with an inflow portion 332 and an outflow portion 334. A lumen 336 preferably extends between an inflow end 338 and an outflow end 340. Proximate the outflow end 340 is preferably a tapered portion 344 having a construction similar to the embodiments described above with respect to FIG. 4. In some embodiments, the shunt 330 includes cylindrical recessed portions along the elongate body to provide enhanced gripping features during deployment and anchoring following implantation within the eye tissue.

The shunts depicted in FIGS. 5 and 6 are preferably constructed of metals, ceramics, or plastics. For example, the shunts 230, 330 can be constructed of titanium and manufactured by conventional sintering, micro machining, laser machining, and/or electrical discharge machining. The shunts of FIGS. 5 and 6 preferably restrict fluid flow in similar manners described above with respect to the embodiments of FIG. 4. Alternatively, where the shunts include a porous body the cross-section and porosity can be calibrated (done to 0.5 micrometers) to control the flow rates of aqueous humor through the shunt. The flow rates through the shunts illustrated in FIGS. 5 and 6 preferably are similar to the rates specified above.

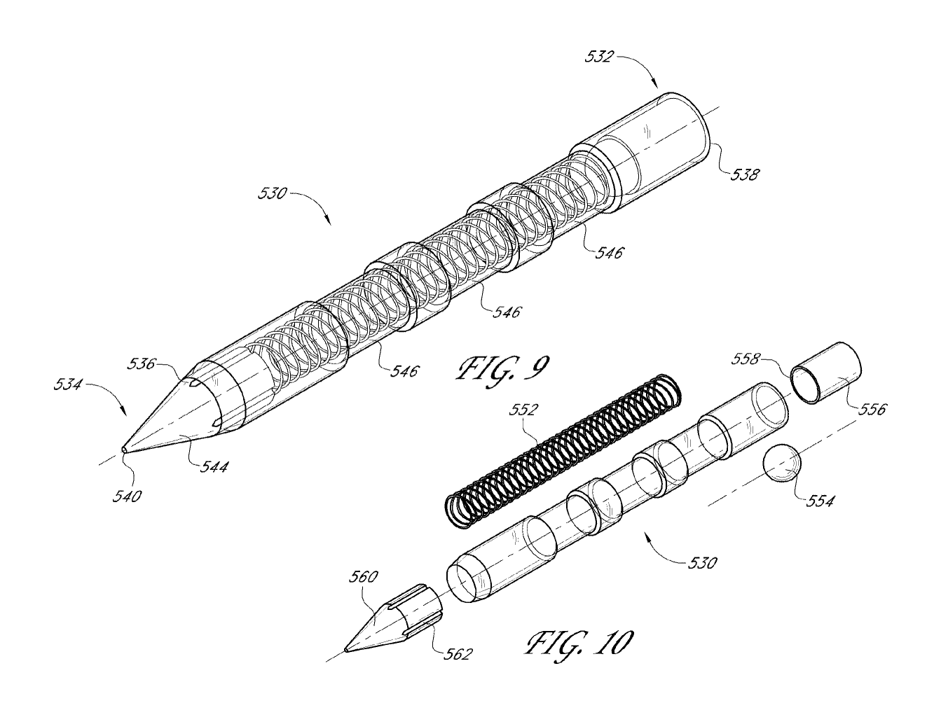

Embodiments Illustrated in FIGS. 7 and 8

FIGS. 7-8 depict embodiments of another shunt 430 having an elongate body with an inflow portion 432 and an outflow portion 434. A lumen 436 preferably extends between an inflow end 438 and an outflow end 440. Although the illustrated embodiment includes just one lumen, other embodiments can include multiple lumens, each including the flow restriction described below.

Proximate the outflow end 440 is preferably a tapered portion 444 that decreases in a radial dimension along a lumen axis 442. In some embodiments, the shunt 430 includes cylindrical recessed portions 446 along the elongate body to provide enhanced gripping features during deployment and anchoring following implantation within the eye tissue. The lumen 436 is preferably configured to accommodate placement therein of a spiral flow channel core 448 that is configured to provide preferred flow restriction.

The core 448 is preferably configured to extend through the lumen 436 between the inflow end 438 and the outflow end 440 and includes a tortuous or spiral flow channel 450 extending generally along the exterior of the core 448. In effect, the spiral flow channel 450 provides an extended path for the flow of fluid between the two ends of the shunt 430 that is greater than a straight lumen extending between the ends of the shunt 430. The extended path provides a greater potential resistance of fluid flow through the shunt without increasing the length of the shunt.

While the core 448 is depicted in FIGS. 7 and 8 as having only a single spiral flow channel 450, the core 448 could have a plurality of spiral flow channels 450 for providing a plurality of flow paths through which fluid may flow through the shunt 430. For example, the core 448 can have two or more spiral flow channels 450. Additionally, the core 448 can also have one or more straight lumens extending through the core 448.

The shunt 430 is preferably manufactured of metals, ceramics, or plastics through conventional micro machining, laser machining, or electrical discharge machining. For example, the shunt 430 can be constructed of titanium, glass, or noble metals. In some embodiments, the core 448 is made of the same material as the body of the shunt 430 while in yet further embodiments, the core 448 includes a material that is different than the body of the shunt 430.

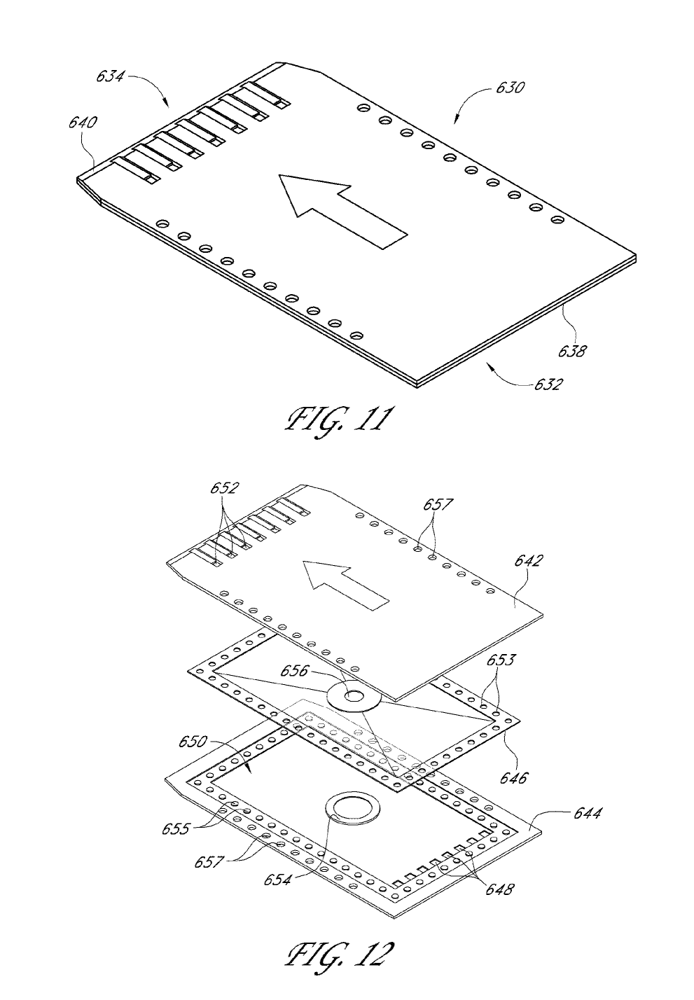

Embodiments Illustrated in FIGS. 9 and 10

FIGS. 9-10 depict embodiments of another shunt 530 having an elongate body with an inflow portion 532 and an outflow portion 534. The shunt 530 preferably includes a lumen 536 that extends between an inflow end 538 and an outflow end 540. The shunt 530 preferably includes a tapered portion 544 at the outflow end 540 that decreases in a radial dimension along a lumen axis 542. In some embodiments, the shunt 530 includes cylindrical recessed portions 546 along the elongate body to provide enhanced gripping features during deployment and anchoring following implantation within the eye tissue.

The shunt 530 is preferably configured to conduct fluid between the anterior chamber and the uveoscleral outflow pathway with the inflow end 538 exposed to the anterior chamber and the outflow end 540 exposed to the suprachoroidal space. The shunt 530 preferably reduces the likelihood of hypotony of the eye by providing a ball-check pressure regulator. For example, when the intraocular pressure exceeds a particular pressure, the ball-check pressure regulator will open and permit fluid to flow between the anterior chamber and the uveoscleral outflow pathway. When the intraocular pressure reaches a second, lower pressure, the ball-check pressure regulator will close and limit or inhibit fluid from being conducted to the uveoscleral outflow pathway. The ball-check pressure regulator will remain closed until the intraocular pressure again reaches the particular pressure, and at which time the ball-check valve will reopen to permit or enhance drainage of fluid to the uveoscleral outflow pathway. Accordingly, the shunt 530 provides drainage of the anterior chamber through the shunt 530 based on the intraocular pressure levels and provides a means for reducing the likelihood for over-draining the anterior chamber and causing hypotony.

The ball-check regulator is preferably configured to be positioned within the lumen 536 of the shunt 530 and includes a luminal spring 552 that is configured to reside within the lumen. The luminal spring 552 is depicted as a coil spring, but the luminal spring 552 can be any type of spring or biasing member that is resilient or reversibly compressible. For example, the spring 552 can comprise Nitinol or other flexible or resilient materials. The ball-check regulator also preferably includes a ball 554 that preferably has a diameter less than the diameter of the lumen 536 of the shunt 530 so as to permit movement of the ball 554 within the lumen 536 and to permit the flow of fluid between the ball 554 and the inner wall of the lumen 536 when the ball 554 resides within the lumen 536. The luminal spring 552 is preferably configured to engage a ball 554 at one end of the luminal spring 552 and move the ball 554 between different positions within the lumen 536.

A ball sleeve 556 is preferably provided within at least a portion of the lumen 536 and is positioned adjacent to the ball 554 opposite the luminal spring 552. For example, FIGS. 9 and 10 depict the ball sleeve 556 positioned adjacent the inflow end 538. The luminal spring 552 is depicted as extending from the outflow portion 534 toward the inflow portion 532 with the ball 554 interposed between one end of the luminal spring 552 and the ball sleeve 556. The portion of the ball sleeve 556 that is adjacent the ball 554 preferably has a lumen that has a diameter less than that of the ball 554 and limits movement of the ball 554 so the ball is unable to pass through the ball sleeve lumen. This end of the ball sleeve 556 preferably provides a ball seat 558 against which the ball 554 can rest when urged against the ball sleeve 556 by the luminal spring 552. In some embodiments, the ball 554 prevents flow when contacting seat of the ball sleeve 556; however, in other embodiments, some restricted flow can occur through the shunt even when the ball 554 rests against the seat. Such flow can occur through one or more parallel flow paths or through one or more relatively small flow paths that extend around the ball 554 and remain open when the ball 554 contacts the seat of the ball sleeve 556.

The shunt 530 also preferably includes a distal taper or cone 560 that is configured to reside at least partially within the lumen 536. The distal cone 560 preferably includes radial flanges 562 that provide a means for securing the cone 560 in place by engaging the inner wall of the lumen 536 while providing a space between the distal cone 560 and the inner wall of the lumen 536. In some embodiments, the distal cone 560 provides radial channels 562 instead of flanges through which the fluid can be conducted. The space between the inner wall of the lumen 536 and the cone 560 or the channels 562 permits fluid conducted through the lumen 536 to exit the shunt by flowing around the distal cone 560.

When the ball-check pressure regulator is assembled, the luminal spring 552 is preferably seated against the distal cone 560 on one end and presses against the ball 554 on the other end with a determined force. The ball 554 is moved against the ball seat 558 of the ball sleeve 556 as a reaction to the force of the luminal spring 552. When the shunt 530 is inserted within the eye with the inflow end 538 exposed to the anterior chamber and the outflow end 540 exposed to the suprachoroidal space, the ball 554 will be exposed to the intraocular pressure of the anterior chamber. The ball 554 will be pressed against the ball seat 558 and limit or inhibit flow of fluid past the ball 554 until the intraocular pressure exerts a force upon the ball 554 that is greater than the force applied by the luminal spring 552. When the luminal spring 552 force is overpowered by the intraocular pressure, the ball 554 will be moved down the lumen 536 away from the ball seat 558, thus permitting fluid to pass around the ball 554, through the lumen 536, and out the outflow portion 534 between the radial flanges 562 of the distal cone 560. When the intraocular pressure drops, the force pressing against the ball 554 will be reduced, and when the force applied on the ball 554 by the intraocular pressure is less than the force applied on the ball 554 by the luminal spring 552, the ball 554 will be moved through the lumen 536 until it is pressed against the ball seat 558, thus stopping the flow of fluid through the lumen 536.

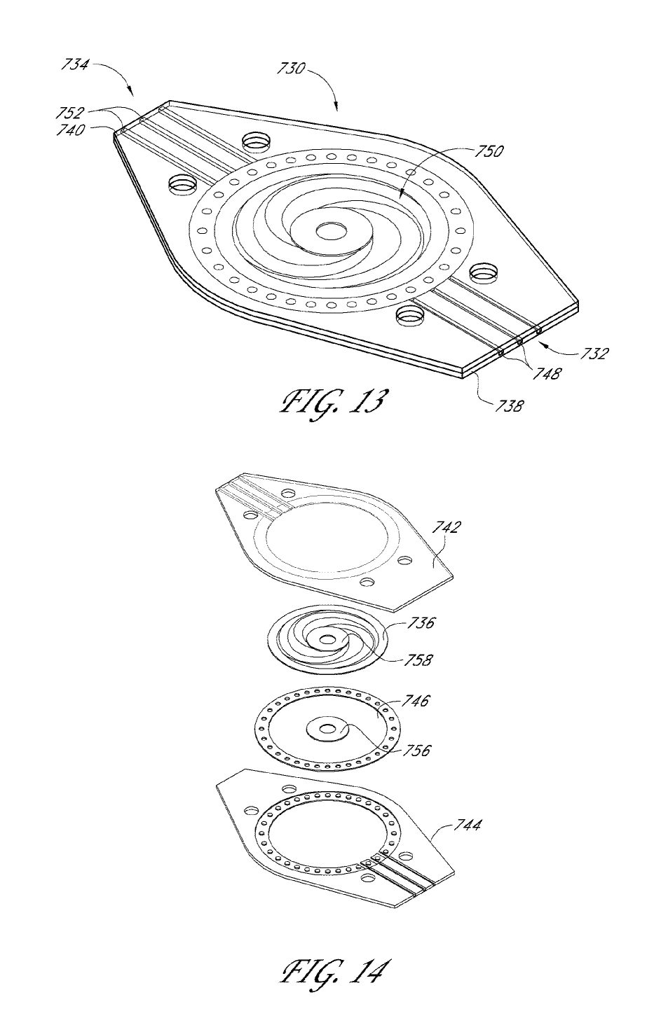

Embodiments Illustrated in FIGS. 11 and 12

FIGS. 11 and 12 illustrate embodiments of a generally flat pressure regulator shunt 630. The shunt 630 preferably includes an inflow portion 632 and an outflow portion 634. The inflow portion 632 preferably includes a plurality of inlets along an inflow end 638, and the outflow portion 634 preferably includes a plurality of outlets along an outflow end 640. The shunt 630 is preferably constructed of three portions: a top portion 642, a bottom portion 644, and a middle portion 646. The top portion 642 and the bottom portion 644 are preferably substantially rigid and provide a housing for the shunt 630. The top portion 642 is engageable with the bottom portion 644 by aligning a plurality of apertures 651 extending along the edges of the portions 642, 644. The two portions 642, 644 can be secured together by glue, solder, or other means for connection the portions. The bottom portion 644 preferably includes inflow apertures 648 that are configured to permit fluid to enter into a chamber 650 formed by the edges of the shunt 630. The top portion 642 preferably includes a plurality of outflow apertures 652 through which fluid can exit the chamber 650 and be discharged from the shunt 630.

Between the two portions 642, 644 is preferably positioned a flexible or resilient middle portion 646. The middle portion 646 is preferably a biased membrane that is biased toward the bottom portion 644 when the shunt 630 is assembled and rests on a membrane seat 654. A plurality of apertures 653 along the edges of the membrane preferably coincides with a plurality of protrusions 655 on the top and bottom portions 642, 644. When the shunt 630 is assembled, the interlocking protrusions 655 and apertures 653 create a seal that reduces the likelihood of fluid from leaking from the chamber 650. The middle portion 646 is preferably constructed of a nitinol sputter deposited silicone membrane. The membrane preferably pressed against the bottom portion 644 and has an aperture 656 extending therethrough. The aperture 656 provides a flow path through which fluid conducted through the shunt 630 can pass when the membrane does not rest on the membrane seat 654.

In operation, the shunt is inserted into the eye with the inflow portion 632 exposed to the anterior chamber and the outflow portion 634 exposed to the uveoscleral outflow pathway. Fluid from the anterior chamber will enter into the inflow apertures 648 and fill the chamber 650 on one side of the membrane of the middle portion 646. Because the middle portion membrane 646 is biased toward the membrane seat 654, the aperture 656 will not permit fluid to flow to the other side of the membrane. When the intraocular pressure reaches an elevated level, the fluid pressure within the chamber 650 will create a force against the membrane 646 and cause the membrane 646 to disengage the membrane seat 654. As the membrane 646 disengages the membrane seat 654, the membrane aperture 656 permits fluid to flow through the membrane 646 into the other side of the chamber 650 and out the outflow apertures 652. The pressure at which the membrane will be deflected from the membrane seat 654 preferably corresponds to acceptable intraocular pressure levels. The large surface area of the membrane 646 will provide for a low tolerance of pressure regulation.

The shunt 630 is preferably implanted following the creation of an incision in the tissue. In some embodiments, the delivery instrument may create the incision for the shunt 630 and deploy the shunt 630 into the incision. In yet other embodiments, the shunt 630 can have a sharpened outflow end 640 and create the incision itself as it is advanced through the tissue.

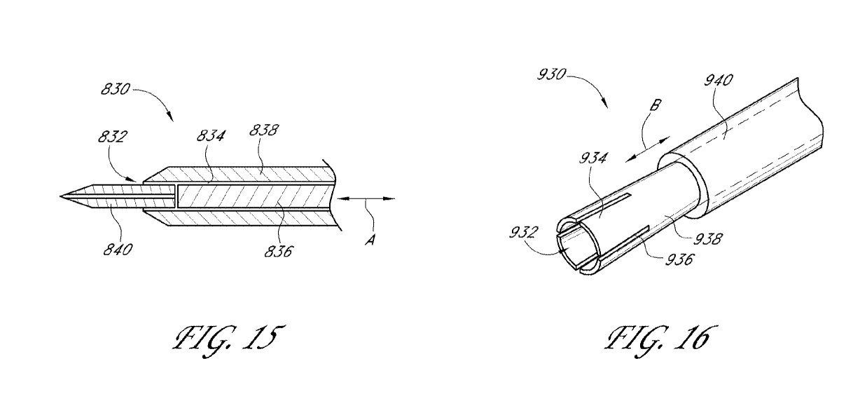

Embodiments Illustrated in FIGS. 13 and 14

FIGS. 13 and 14 depict a shunt 730 that operates under similar principles as that of the embodiments depicted in FIGS. 11 and 12. The shunt has an inflow portion 732 and an outflow portion 734. The inflow portion 732 includes an inflow end 738 and inflow apertures 748. The outflow portion 734 includes an outflow end 740 and outflow apertures 752. The inflow apertures 748 and the outflow apertures 752 are in fluid communication with a shunt chamber 750. The shunt 730 preferably includes four portions: a top portion 742, a spring or biasing portion 736, a membrane portion 746, and a bottom portion 744. When the shunt is assembled, the sprint or biasing portion 736 preferably presses the membrane portion 746 against the bottom portion 744, thus restricting the fluid communication through the shunt 730. When the intraocular pressure reaches a certain level, the resultant force exerted against the membrane portion 746 will exceed that of the spring or biasing portion 736 and cause the membrane portion 746 to disengage the bottom portion 744. When the membrane portion 746 is not pressing against the bottom portion 744, an aperture 756 will permit fluid to flow through the membrane 746 and through an aperture 758 in the spring or biasing portion 736. During the period of flowing fluid, the aqueous humor will flow through the chamber 750 and out the shunt 730 through the outflow apertures 752.

In some embodiments of the illustrated shunts in FIGS. 11-14, an intraocular pressure regulator is provided having an inlet portion that provide at least one ingress flow path that include one or more influent openings. The openings preferably have a total cross-sectional flow area and communicate with an interior chamber within the shunt. In some embodiments, the shunts include an outlet portion that provides an egress flow path that has one or more effluent openings. In yet further embodiments, the shunts have a pressure regulation valve that includes a deflectable plate with a surface area exposed to fluid within the interior chamber. The surface area preferably is substantially greater than the total cross-sectional flow area. The valve is preferably located between the interior chamber and one or more effluent openings such that movement of the deflectable plate regulates flow from the interior chamber to the one or more effluent openings. The plate preferably extends in a direction generally parallel to the inlet flow path and to the outlet flow path.

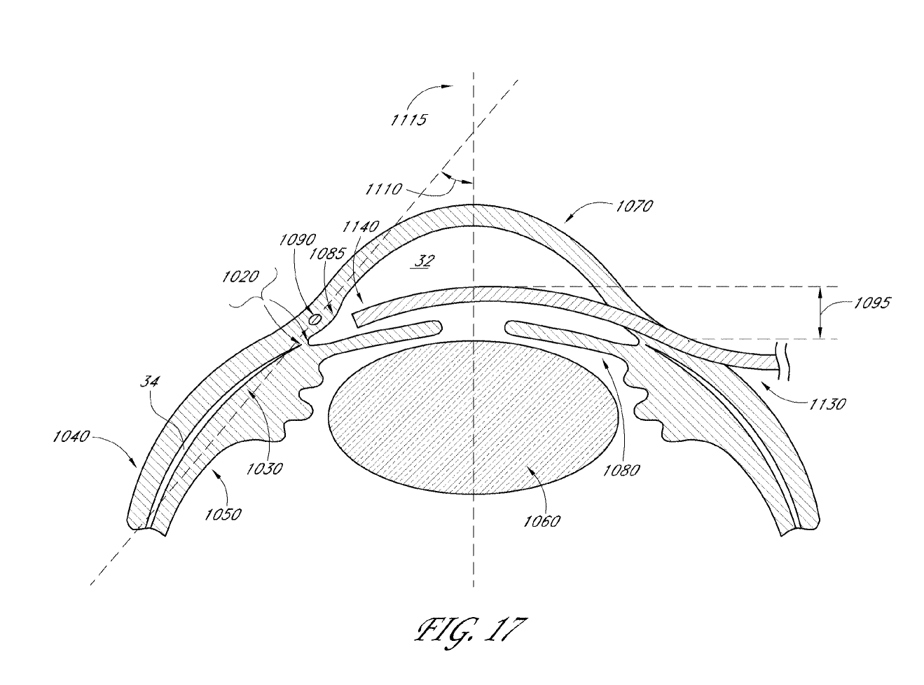

Embodiments Illustrated in FIG. 15

FIG. 15 illustrates one embodiment of a delivery instrument 830 that can be used with embodiments of shunts described herein. The delivery instrument 830 preferably includes an open distal end 832 with a lumen 834 extending therethrough. Positioned within the lumen 834 is preferably a pusher tube 836 that is axially movable within the lumen 834, as indicated by the arrows A. A wall 838 of the delivery instrument 830 preferably extends beyond pusher tube 836 to accommodate placement within the lumen 834 of a shunt 840. The shunt 840 can be secured in position. For example, the shunt 840 can be secured by viscoelastic or mechanical interlock with the pusher tube 836 or wall 838. When the shunt is brought into position adjacent the tissue in the anterior chamber angle, the pusher tube 836 is advanced axially toward the open distal end 832 of the delivery instrument 830. As the pusher tube 836 is advanced, the shunt 840 is also advanced. When the shunt 840 is advanced through the tissue and such that it is no longer in the lumen 834 of the delivery instrument 830, the delivery instrument 830 is retracted, leaving the shunt 840 in the eye tissue.

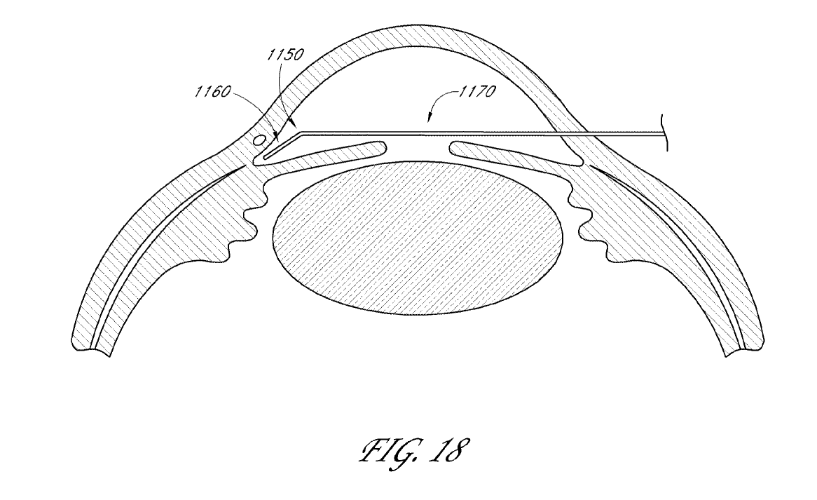

Embodiments Illustrated in FIG. 16

FIG. 16 illustrates another embodiment of a delivery instrument 930 that can be used with embodiments of shunts described herein. The delivery instrument 930 preferably has an open distal end 932 that is configured to receive a shunt (not shown). The delivery instrument 930 preferably has a plurality of prongs 934 that are separated circumferentially by axially-extending slots 936 in an inner cylinder 938 of the delivery instrument. The prongs 934 are preferably slightly biased radially outward and are able to be forced radially inward to grasp a shunt that resides within the open distal end 932. A slider tube 940 is preferably positioned around the inner cylinder 938 and has an inner diameter that is slightly larger than the outer diameter of the inner cylinder 938. The slider tube 940 is preferably axially movable over the inner cylinder 938 in the direction of the arrows B. As the slider tube 940 is advanced over the prongs 934, the prongs 934 are forced radially inward and the gaps created by the slots 936 are reduced. As the prongs 934 are forced radially inward, the inner diameter of the inner cylinder 938 is reduced, and the prongs 934 can firmly grasp a shunt that is positioned therein. When the shunt is properly positioned within the eye tissue, the slider tube 940 is withdrawn to permit the prongs 934 to expand radially outwardly, and the shunt is released from the grip of the prongs 934. The delivery instrument 930 is then removed from the eye. If the shunt needs to be repositioned, the delivery instrument 930 can re-grip the shunt by placing the prongs 934 over the shunt and advancing the slider tuber 940 over the prongs. The shunt can be release following its repositioning or orienting in the same manner as described above. If multiple shunts are required, a new shunt can be inserted into the delivery instrument and deployed in the same manner as described above.

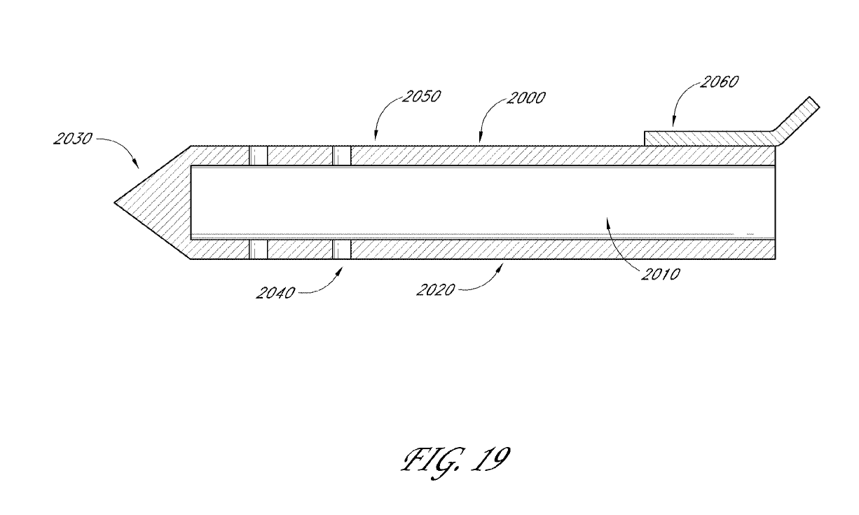

Embodiments Illustrated in FIGS. 17 and 18

FIG. 17 shows a meridional section of the anterior segment of the human eye and illustrates another embodiment of a delivery instrument 1130 that can be used with embodiments of shunts described herein. In FIG. 17, arrows 1020 show the fibrous attachment zone of the ciliary muscle 1030 to the sclera 1040. The ciliary muscle is part of the choroid 1050. The suprachoroidal space 34 is the interface between the choroid and the sclera. Other structures in the eye include the lens 1060, the cornea 1070, the anterior chamber 32, the iris 1080, and Schlemm's canal 1090.

In some embodiments, it is desirable to implant a shunt through the fibrous attachment zone, thus connecting the anterior chamber to the uveoscleral outflow pathway, in order to reduce the intraocular pressure in glaucomatous patients. In some embodiments, it is desirable to deliver the shunt with a device that traverses the eye internally (ab interno), through a small incision in the limbus.

The delivery instrument/shunt assembly must be passed between the iris and the cornea to reach the iridocorneal angle. Therefore, the height of the delivery instrument/shunt assembly (dimension 1095 in FIG. 17) must be less than about 3 mm, and preferably less than 2 mm.

The suprachoroidal space between the choroid and the sclera forms an angle 1110 of about 55 degrees with the optical axis 1115 of the eye. This angle, in addition to the height requirement described in the preceding paragraph, are features to consider in the geometrical design of the delivery instrument/shunt assembly.

The overall geometry of the system makes it advantageous that the delivery instrument 1130 incorporates a distal curvature 1140, as shown in FIG. 17, or a distal angle 1150, as shown in FIG. 18. The distal curvature (FIG. 17) is expected to pass more smoothly through the incision. However, the shunt should be curved or flexible in this case. Alternatively, in the design of FIG. 18, the shunt may be mounted on the straight segment of the delivery instrument, distal of the “elbow” or angle 1150. In this case, the shunt may be straight and relatively inflexible, and the delivery instrument can incorporate a deployment mechanism that is flexible enough to advance through the angle. In some embodiments, the shunt is a rigid tube, provided that the shunt is no longer than the length of the distal segment 1160.

The distal curvature 1140 of delivery instrument 1130 may be characterized as a radius of approximately 10 to 30 mm, and preferably about 20 mm. The distal angle of the delivery instrument depicted in FIG. 18 may be characterized as approximately 90 to 180 degrees relative to the proximal segment 1170 of the delivery instrument, and preferably about 145 degrees. The angle incorporates a small radius of curvature at the “elbow” so as to make a smooth transition from the proximal segment 1170 of the delivery instrument to the distal segment 1160. The length of the distal segment 1160 may be approximately 0.5 to 7 mm, and preferably about 2 to 3 mm.

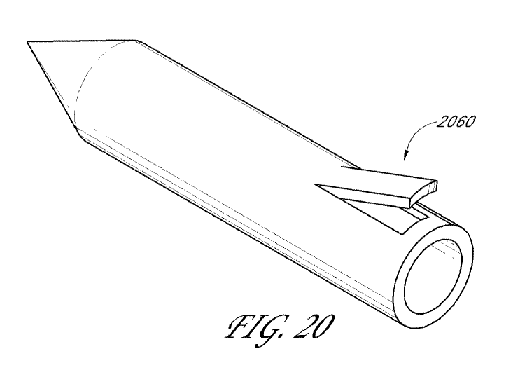

Embodiments Illustrated in FIGS. 19 and 20

FIG. 19 illustrates another embodiment of a shunt 2000 that is operable to drain fluid from the anterior chamber to the suprachoroidal space. The shunt can include one or more lumens 2010, a circumferential wall 2020, and a tip 2030. The tip may be pointed (for pushing through resistant tissue), or rounded (to be incapable of penetrating tough tissue such as the sclera). One or more sideholes 2040 in the wall permit the egress of aqueous fluid flowing from the anterior chamber through lumen(s) 2010. The tip 2030 may be comprised of thermoplastic material such as polyurethane or Pebax or polymethylmethacrylate or polyimide, or elastomeric material such as silicone, or metal material such as titanium, steel, or nitinol. The tip 2030 may be attached to the longitudinal body section 2050 of the shunt by molding, or adhesive bonding, or thermal bonding. The longitudinal body may be comprised of thermoplastic material such as polyurethane or Pebax or polymethylmethacrylate or polyimide, or elastomeric material such as silicone, or metal material such as titanium, steel, or nitinol. The body material is preferably flexible, such as polyurethane or Pebax or silicone. However, it may be comprised of rigid material such as polymethylmethacrylate or metal. In this case, the shunt may be made flexible by creating one or more indentations, or by etching or machining or laser processing a relief pattern in the wall of the shunt, such is known in the art of design and fabrication of shunts for the coronary arteries. The shunt does not need to provide a solid tubular conduit between the anterior chamber and the suprachoroidal space, as the shunt will be surrounded by tissue, and the fluid flow will thus be constrained within the tubular envelope created by the shunt.

In some embodiments, the flexible shunt has an outer diameter of approximately 0.1 to 2.0 mm diameter, preferably about 0.4 mm. The length of the shunt is approximately 0.5 to 7 mm, preferably about 2 to 4 mm.

The shunt may also incorporate fixation features 2060, such as flexible radial extensions. The extensions may be separate pieces attached to the shunt, or may be formed by slitting the shunt wall, and thermally forming or mechanically deforming the extensions radially outward, as shown in FIG. 20. If the extensions 2060 are separate pieces, they may be comprised of flexible material such as nitinol or polyimide. The extensions 2060 may be located at the anterior or posterior ends of the shunt, or both, to prevent extrusion of the shunt from its intended location. The flexibility of the fixation features will facilitate entry through the corneal incision, and also through the ciliary muscle attachment tissue.

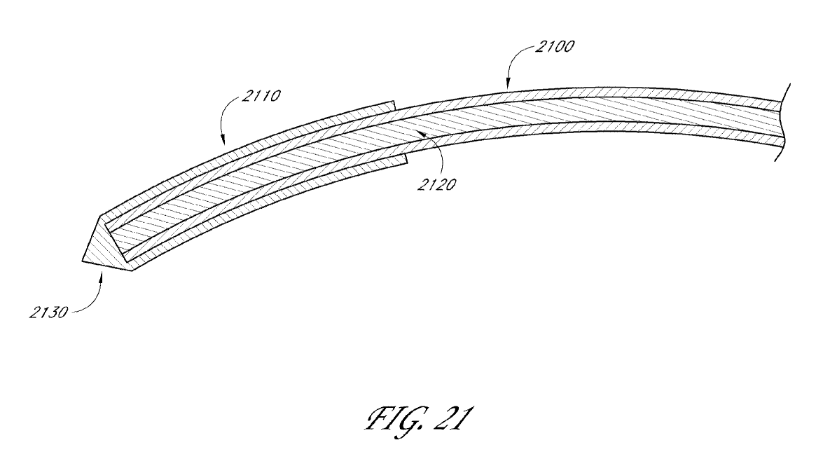

Embodiments Illustrated in FIG. 21

FIG. 21 shows another embodiment of a system that can be used to perform a variety of methods or procedures. The curved shaft of a delivery instrument 2100 can be hollow, and the shunt 2110 can be slidably mounted on the outer diameter of the delivery instrument. The shunt is preferably flexible. A flexible, slidable stylet 2120 can be inserted through the shaft of the delivery instrument, and pushes against the inner wall of shunt tip 2130. The stylet 2120 can be comprised of a flexible material with a high modulus of elasticity, such as stainless steel, and preferably nitinol. The proximal end of the delivery instrument is not shown, but provides for a sliding mechanism to advance and retract the stylet 2120 by the operator. The mechanism may be incorporated into a handle, such as the push-pull controls in the handles of electrophysiology catheters known in the art; or the proximal end of the stylet 2120 may extend outward from the proximal end of the shaft, such that the operator may grasp it directly to push and pull it.

In some embodiments, during clinical use, the shunt and shaft assembly can be advanced together through the limbus, across the iris, and through the ciliary muscle until the shunt tip is located in the suprachoroidal space. The operator then simultaneously pushes on the stylet 2120 while pulling back on the delivery instrument 2100, such that the shunt tip maintains its location in the suprachoroidal space, the shunt 2110 is released distally from the delivery instrument 2100, and the delivery instrument 2100 is retracted proximally. At this point, the shunt 2110 is still riding on the distal end of the stylet 2120. The next step is to withdraw the stylet 2120, leaving the shunt 2110 in place in the tissue. Finally, the delivery instrument 2100 is withdrawn from the anterior chamber through the incision.

A shunt and delivery instrument assembly, including a flexible stylet, similar to that shown in FIG. 21 can also be used in conjunction with the angled delivery instrument of FIG. 18 and a rigid tube shunt, provided the shunt is no longer than the length of the distal segment 1160. The operation is similar to that described in the preceding paragraph.

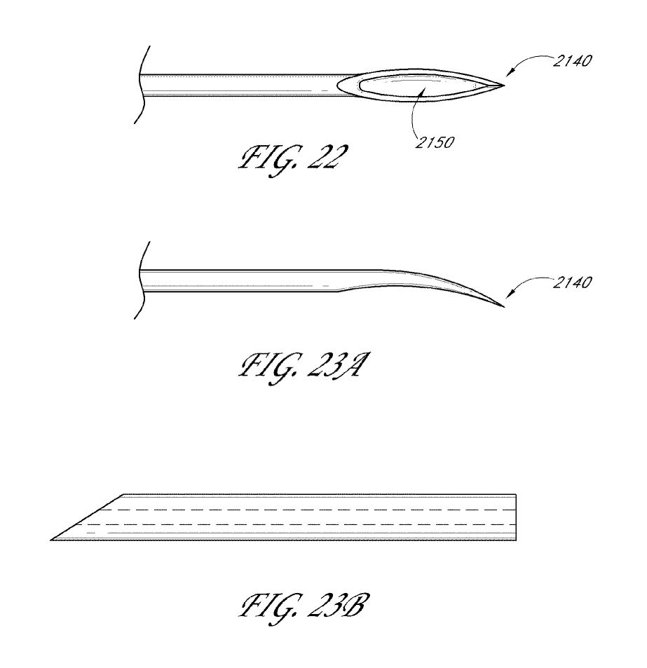

Embodiments Illustrated in FIGS. 22 and 23

FIGS. 22 and 23 show an example of a delivery instrument for a shunt. In some embodiments, the shunt is delivered through a needle with a cutting tip 2140. The shunt can be loaded inside of the shaft of the needle for delivery through the eye. The needle can be curved on the side of the needle opposite to the beveled opening 2150, as illustrated in FIG. 23(a). This allows the curved part of the needle to take a “downward” direction without appreciably affecting the effective height of the device. This geometry can be advantageous for passage through the anterior chamber between the iris and the cornea. At the same time, the curve permits the sharp tip of the needle to follow the angle of the ciliary muscle/ sclera interface. Further, the design of the curved tip as shown in FIG. 23(a) can limit the depth of the dissection of the ciliary muscle from the sclera to the minimum depth necessary to cut through the fibrous attachment tissue. This depth is estimated to be less than about 0.5 mm. In addition, the curvature of the tip act as a baffle to redirect the shunt as it is pushed distally outward through the needle. In other embodiments, the needle cutting tip is straight, as illustrated in FIG. 23(b).

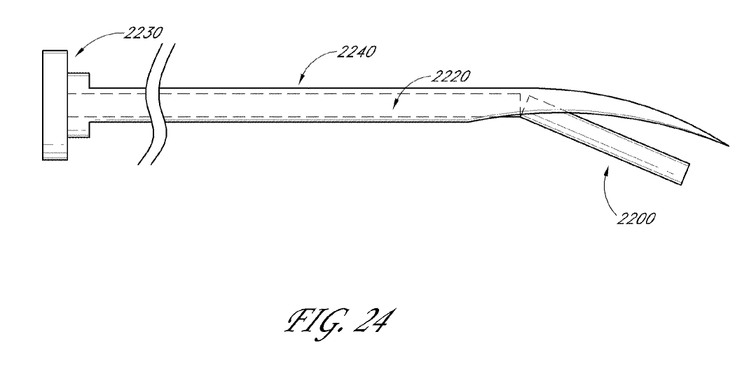

Embodiments Illustrated in FIG. 24

FIG. 24 shows another embodiment of a system that can be used to perform a variety of methods or procedures. The shunt 2200 is deflected “downward” at an angle that parallels the suprachoroidal space. The depth of insertion can be determined by the length of the pushrod 2220, whose travel can be limited by the stop 2230. It is preferred that the pushrod ends at the proximal edge of the opening of the needle 2240. In this way, the shunt will not be pushed below the anterior surface of the ciliary muscle.

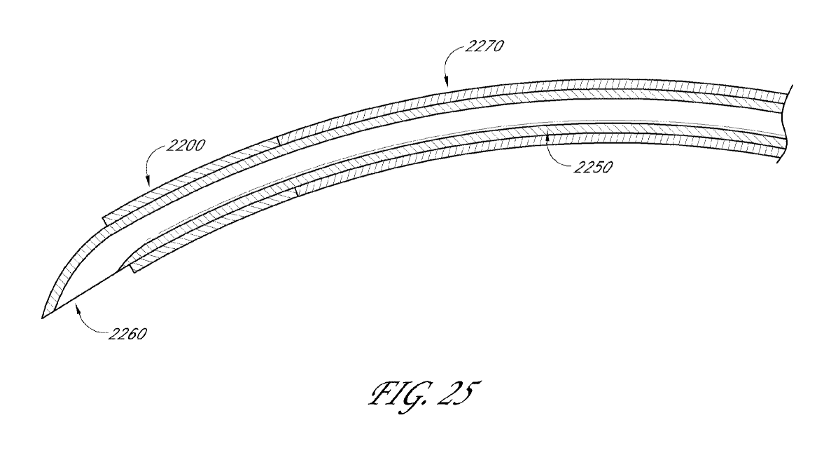

Embodiments Illustrated in FIG. 25

FIG. 25 shows another embodiment of a system that can be used to perform a variety of methods or procedures. In the illustrated embodiment, the shunt 2200 is mounted on a curved or angled shaft 2250. The shaft 2250 can be tubular, and the distal end 2260 can be sharpened. The shunt 2200 can be curved with approximately the same radius as the delivery device, so that the shunt can be relatively stiff and still slide along the shaft. In some embodiments, a pusher tube 2270 causes the shunt to slide distally along the shaft and be released. In operation in some embodiments, the sharpened end 2260 makes an incision in the fibrous tissue attaching the ciliary muscle and the sclera. In some embodiments, the distance between the sharpened tip 2260 and the distal end of the shunt determines how deeply the tissue may be incised. After making the cut, the operator can advance the pusher tube 2270 while holding the mounting shaft 2250 fixed. This action causes the shunt 2200 to be advanced into the incision. The distance of shunt advance can be determined by the length of the pusher tube 2270, whose travel can be limited by a stop, as depicted in FIG. 24.

Further embodiments of the invention incorporate injection of viscoelastic through the shunt in order to accomplish posterior dissection of the suprachoroidal tissue, thereby creating a volumetric chamber or reservoir for aqueous humor.

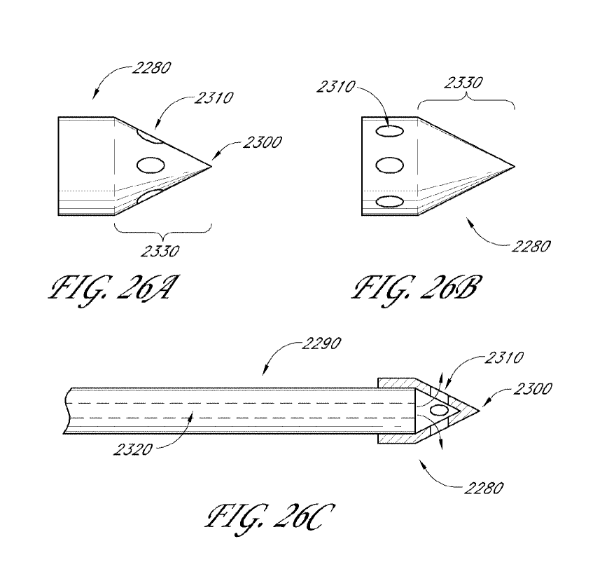

Embodiments Illustrated in FIG. 26

FIG. 26 illustrates various embodiments of a cap 2280 for a shunt 2290 that is operable to drain fluid from the anterior chamber to the suprachoroidal space. The cap 2280 can include a tissue-piercing end 2300 and one or more outlet openings 2310. Each of the one or more outlet openings 2310 can communicate with at least one of the one or more lumens 2320. In some embodiments cap can have a conically shaped tip 2330 with a plurality of outlet openings 2310 disposed proximal of the tip's distal end. In other embodiments, the cap can have a tapered angle tip 2330. The tip 2330 can be sufficiently sharp to pierce eye tissue near the scleral spur of the eye. The tip also can be sufficiently blunt so as not to substantially penetrate scleral tissue of the eye. In some embodiments, the conically shaped tip 2330 facilitates delivery of the shunt to the desired location. In some embodiments, the cap 2280 has an outlet opening 2310 on a side surface to allow fluid flow. In the embodiment illustrated in FIG. 26a, there is a plurality of outlet openings 2310 on the conical surface of the cap. In the embodiment illustrated in FIG. 26b, the cap has a plurality of outlet openings 2310 on a side surface to allow fluid flow. The openings 2310 on the cap can facilitate fluid flow through the shunt. The openings 2310 may provide an alternate route for fluid flow which is beneficial in case the primary outflow portion of the shunt becomes blocked.

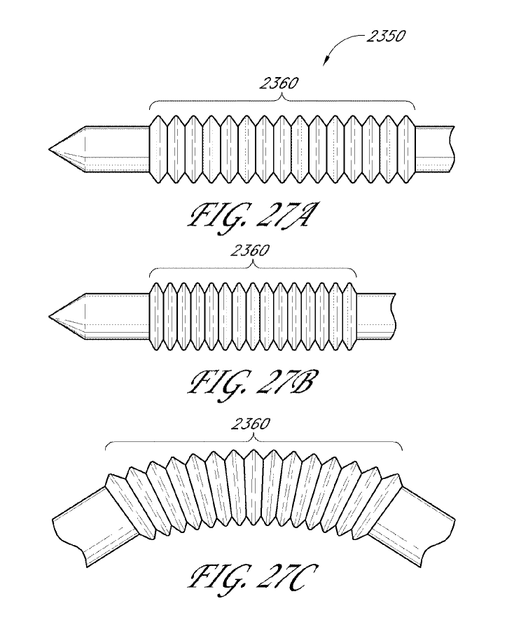

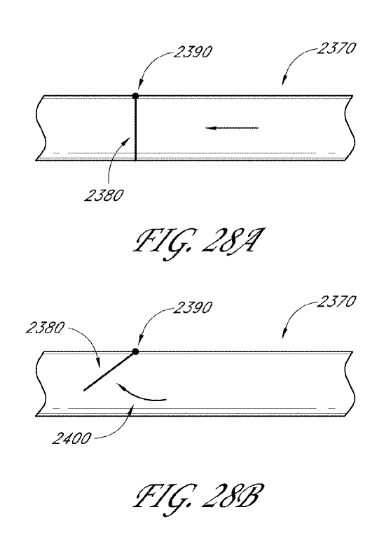

Embodiments Illustrated in FIG. 27

FIG. 27 shows another embodiment of a system that can be used to perform a variety of methods or procedures. The shunt 2350 illustrated in FIG. 27 has a portion 2360 which has an accordion-like structure. The accordion-like structure provides flexibility. FIG. 27(a) depicts the accordion-like portion 2360 in an expanded configuration. FIG. 27(b) depicts the accordion-like portion 2360 in a compressed configuration. FIG. 27(c) depicts the accordion-like portion 2360 in a curved or bended configuration.

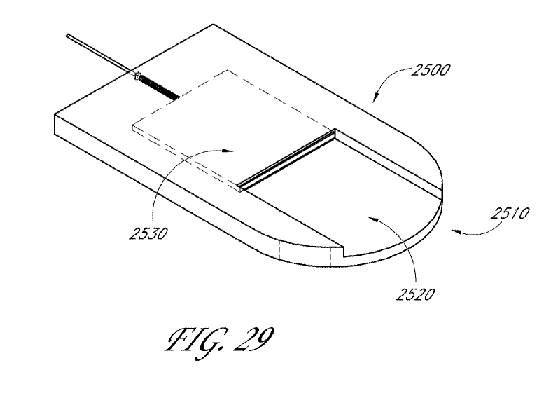

Embodiments Illustrated in FIG. 28

FIG. 28 illustrates another embodiment of a shunt 2370 that is operable to drain fluid from the anterior chamber to the suprachoroidal space. In the illustrated embodiment, the shunt 2370 has a reed-type valve 2380 to regulate flow. One end 2390 of the reed valve 2380 may be fixed to a portion of the shunt. The body of the reed valve 2380 is capable of being deflected 2400 in order to allow flow. The reed valve 2380 illustrated in FIG. 28a is shown in a closed configuration. Pressure from fluid in the anterior chamber can deflect the body of the reed valve 2380, thereby causing the valve to open, as depicted in FIG. 28b.

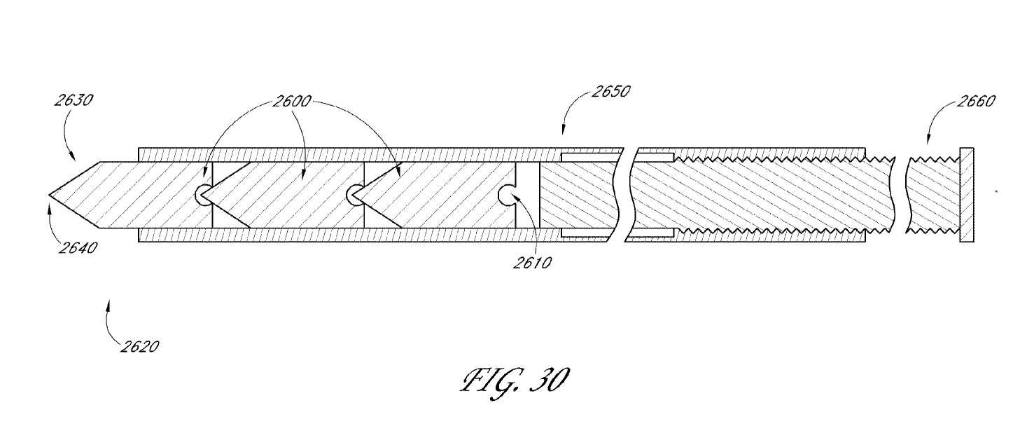

Embodiments Illustrated in FIG. 29

FIG. 29 shows another embodiment of a system that can be used to perform a variety of methods or procedures. In the illustrated embodiment, a delivery instrument includes a distal end 2500 having a spatula shape. The spatula shape can have a sharpened forward edge 2510. The spatula shape can include a recess 2520 to contain the shunt. The recess can include a pusher 2530 or other suitable means to push out or eject the shunt.

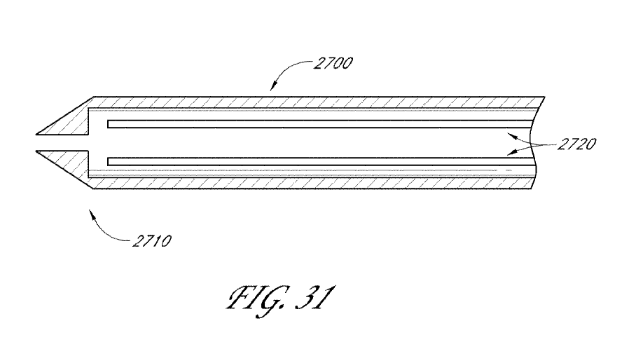

Embodiments Illustrated in FIG. 30

FIG. 30 shows another embodiment of a system that can be used to perform a variety of methods or procedures. Multiple shunts 2600 are configured to be delivered during a single procedure. In the illustrated embodiment, the shunts 2600 are arranged tandemly. The shunt can include a tip protector 2610 at one end. The tip protector 2610 can comprise a recess shaped to receive and protect, for example, the tip 2620 of an adjacent shunt. The tip protector 2610 is shaped to contact the sides 2630 of the conical tip while protecting the more tapered tip, or end 2640, from impact. The delivery instrument 2650 can include a pusher 2660 or other suitable means to push out or eject each shunt 2600.

Embodiments Illustrated in FIG. 31

FIG. 31 shows another embodiment of a system that can be used to perform a variety of methods or procedures. Delivery of the shunt 2700 is achieved by applying a driving force at or near the distal end 2710 of the shunt 2700 using, for example, a pusher 2720. The driving force can be a pulling or a pushing applied to the distal end 2710 of the shunt 2700.

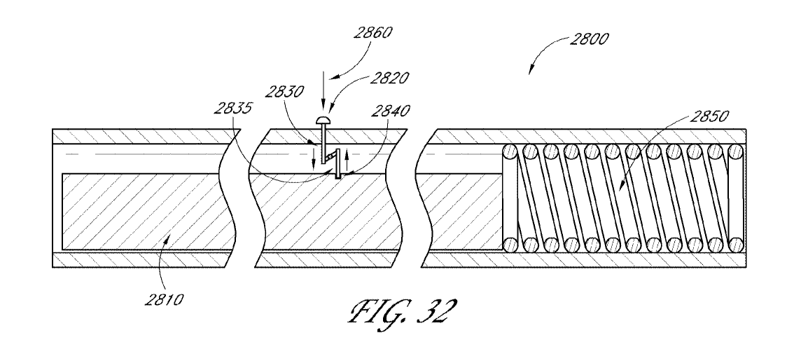

Embodiments Illustrated in FIG. 32

FIG. 32 shows another embodiment of a system 2800 that can be used to perform a variety of methods or procedures. A spring-loaded pusher system 2800 can be used for delivery of a shunt. The spring-loaded pusher 2810 preferably includes a button 2820 operably connected to a hinged rod device 2830. The distal portion 2835 of the hinged rod device 2830 engages a depression 2840 in the surface of the pusher 2810, keeping the spring 2850 of the pusher 2810 in a compressed conformation. When the user pushes downwards 2860 on the button 2820, the distal portion 2835 of the hinged rod device 2830 is disengaged from the depression 2840, thereby allowing the spring 2850 to decompress, thereby advancing the pusher 2810 forward.

Embodiments Illustrated in FIG. 33

FIG. 33 shows another embodiment of a system that can be used to perform a variety of methods or procedures. In the illustrated embodiment, an over-the-wire system is used to deliver the shunt 2900. Preferably, the wire 2910 is self-trephinating. The wire can function as a trocar. The wire can be superelastic, flexible, or relatively inflexible with respect to the shunt. The wire can be pre-formed to have a certain shape. The wire can be curved. The wire can have shape memory, or be elastic. In some embodiments, the wire is a pull wire. The wire can be a steerable catheter.

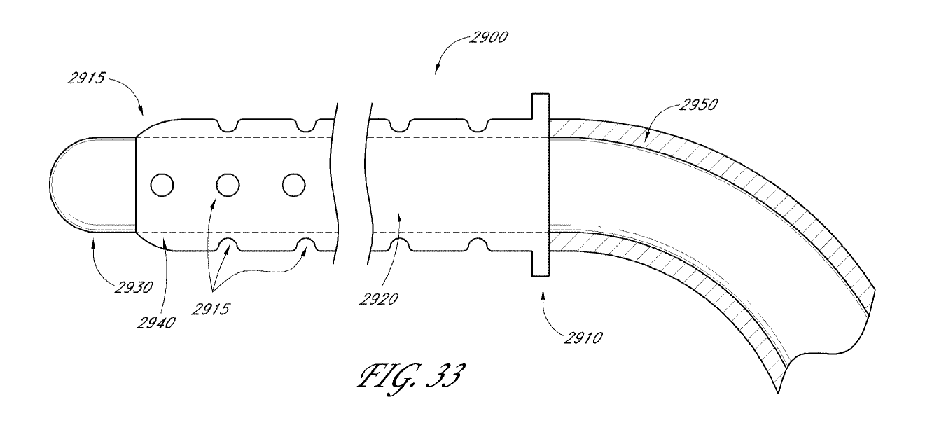

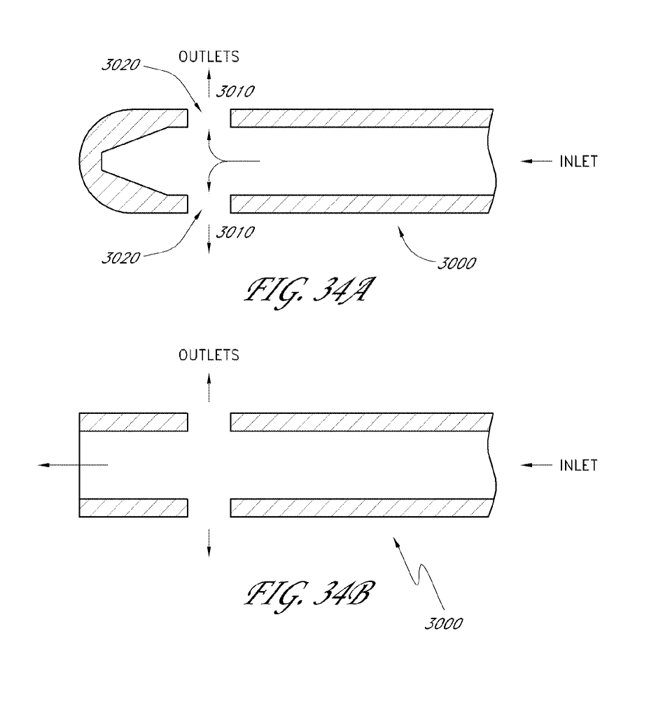

Embodiments Illustrated in FIGS. 34-36

FIGS. 34-36 illustrate other embodiments of a shunt that is operable to drain fluid from the anterior chamber to the suprachoroidal space. In the embodiments illustrated in FIGS. 34A, 34B and 35, the shunt 3000 has an outflow configuration 3010 wherein the flow exits normal (+/−90°) from the axis of the shunt through a sideport exit hole 3020. This outflow configuration can prevent adhesion and/or encapsulation of the tissues that make up the supraciliary or suprachoroidal space (e.g., choroid and sclera and any other membranes within) by, for example, the fluid pressure created, and/or a rinsing effect. As such, the outflow pathway is kept clear and unobstructed. There is a long history of device encapsulation within the eye and eventual blockage of flow. In addition, in this outflow configuration, the flow can exit and directly impinge the tissues that form the uveoscleral outflow pathway. This flow can push and hold the surrounding tissue away from the stent, thereby preventing tissue adhesion to the shunt 3000 at the location of the fluid path. The flow can also help to create a stenting effect, i.e., holding the space open and enlarging. In some embodiments, the stenting can facilitate absorption into the choroid and/or the sclera by increasing the contact area between the pool of aqueous humor and the tissues. The sideport exit holes 3020 can prevent tissues and cells from accumulating in an axial hole during the insertion operation, i.e., the scraping/snowplowing of cells/tissues that could get lodged in the tip and block flow.

In the embodiment illustrated in FIG. 35, the shunt 3000 has an outflow configuration 3100 wherein the flow exits the device not axially, nor at a 90° angle to the device main axis, but at an angle that bisects the two options, i.e., 30-60°. This outflow configuration can help to prevent tissue adhesion and provide the other benefits described above. In addition, this outflow configuration allows the flow to exit the shunt without the slowdown that occurs when it is turned at a 90° angle. This outflow configuration can provide a greater opportunity for the flow to be directed deeper into the suprachoroidal space. In some embodiments, this outflow.

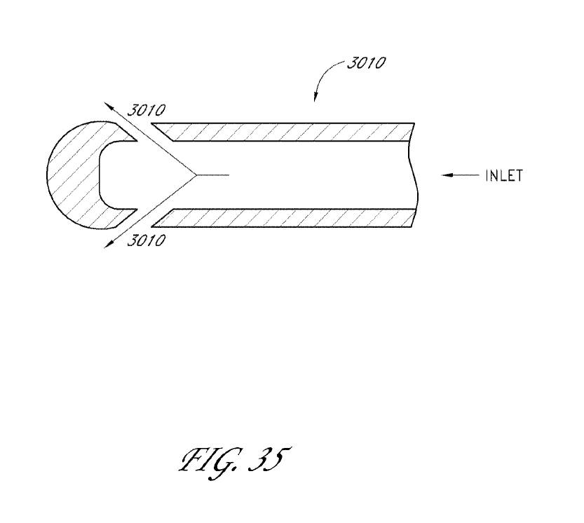

The outflow configuration shown in FIGS. 34A, 34B and 35 can be combined with one or more axial outlets, as shown in FIG. 36. In the embodiment illustrated in FIG. 36, the shunt 3000 has a combination of axial 3200 and sideport 3300 flow. In this embodiment, the relative sizes of the ports 3400, 3500 can be varied to achieve the correct balance between the two flow directions. For example, the axial flow lumen can be sized down so that the side port holes 3400 receive an adequate amount of flow to realize the advantages of side port flow 3300 without sacrificing axial flow 3200 and maximal flow penetration deep into the uveoscleral outflow pathway.

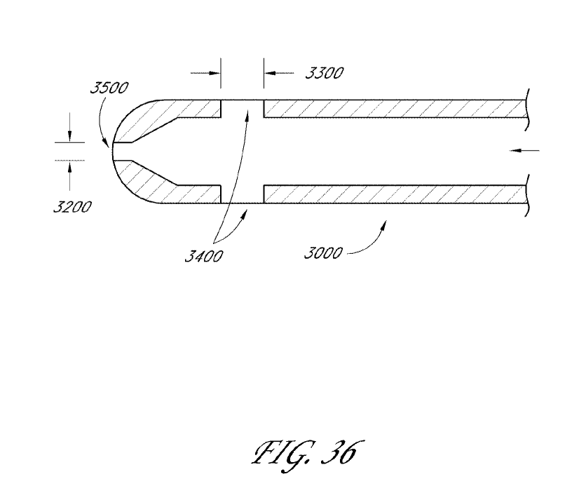

Embodiments Illustrated in FIG. 37

FIG. 37 shows another embodiment of a system that can be used to perform a variety of methods or procedures. The shunt 3600 illustrated in FIG. 37 has a solid and rounded tip with (FIG. 37A) or without (FIG. 37B) a central lumen 3800. The solid and rounded tip 3700 can ease insertion into the uveoscleral outflow pathway.

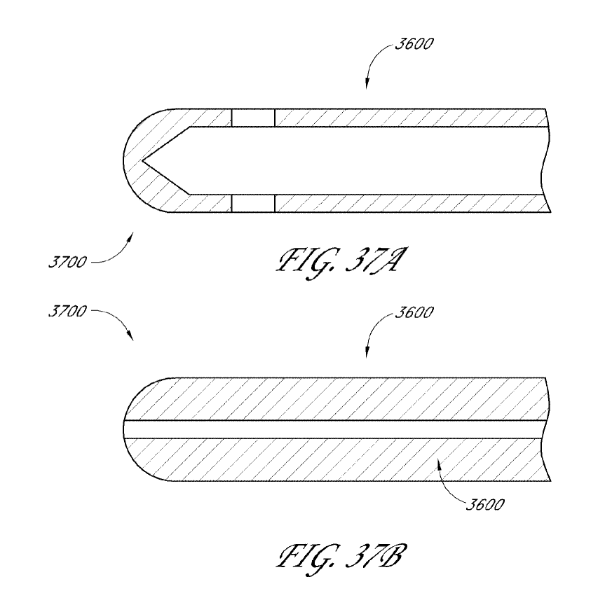

Embodiments Illustrated in FIG. 38

FIG. 38 shows another embodiment of a system that can be used to perform a variety of methods or procedures. The shunt 3900 illustrated in FIG. 38 has a reduced diameter B at the point where the fluid exits the stent, compared with the inlet orifice diameter A. The reduced diameter B can result in an increased fluid velocity V_{B}, compared to the fluid inlet velocity V_{A}. The increased fluid velocity can help to keep tissue at bay, thereby preventing adhesion to the shunt. The increased fluid velocity can also create space for absorption of fluid into the choroid and sclera. The increased fluid velocity can also cause deeper penetration of the fluid once it exits the shunt.

Embodiments Illustrated in FIG. 39

FIG. 39 shows another embodiment of a system that can be used to perform a variety of methods or procedures. The shunt 4000 illustrated in FIG. 39 has sharp edges and corners 4010. The sharp edges and corners 4010 can prevent cell proliferation as can be seen on intra-ocular lens (IOLs) implants.

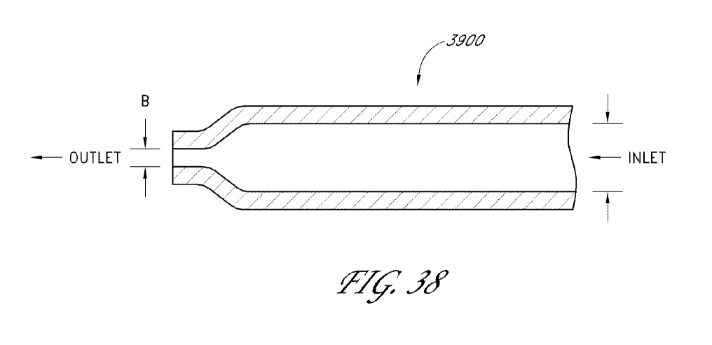

Embodiments Illustrated in FIG. 40

FIG. 40 shows another embodiment of a system that can be used to perform a variety of methods or procedures. A matrix, or grating 4100 can be positioned within the uveoscleral outflow pathway to create, and hold open a space between the ciliary muscle bundles, or the choroid and the sclera, into which fluid can flow. The grating 4100 can effectively decrease or essentially eliminate the resistance that the fluid would encounter upon entering the uveoscleral outflow pathway from a single or double entry point from the anterior chamber, and establish open fluid communication, or, in longer embodiments, contact to a large surface area of choroid and/or sclera for absorption and dissipation. The grating 4100 can be made from a number of biocompatible materials such as, for example, metals like gold, platinum, tantalum, titanium, etc., or from a biocompatible polymer such as silicone, PMMA, polyimide, polyether sulfone (PES), styrene-b-isobutylene-b-styrene (SIBS), ceramic, or from a combination of materials, such as a coating, plating, or coextrusion of one of the mentioned materials with another one of the mentioned or other materials. The grating 4100 can be separate, or integral with a shunt that establishes a patent opening from the anterior chamber 4105 to the suprachoroidal space 4110. As shown in FIG. 40B, the grating 4100 can be injected or otherwise placed through a small opening such as a tube 4120 and unfold or otherwise expand once in the suprachoroidal space so that it can be delivered ab interno. In other embodiments, the grating can be placed ab externo.

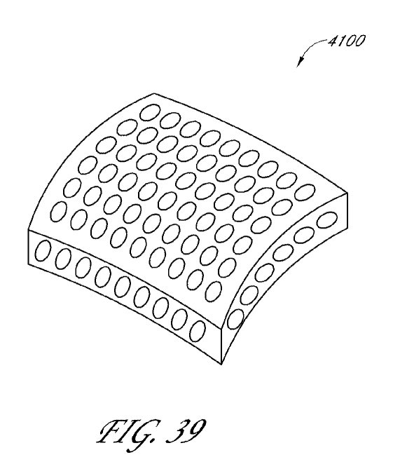

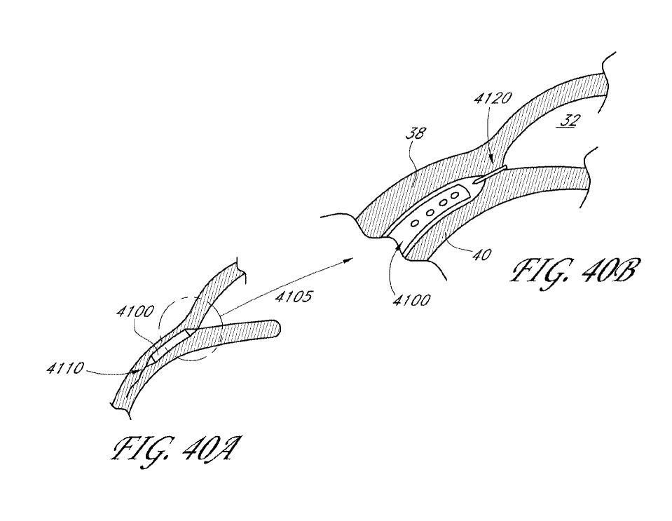

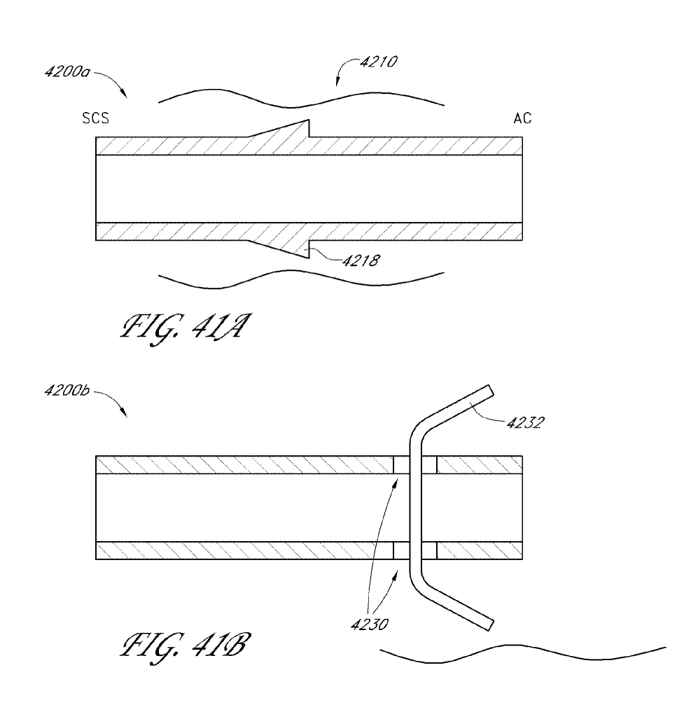

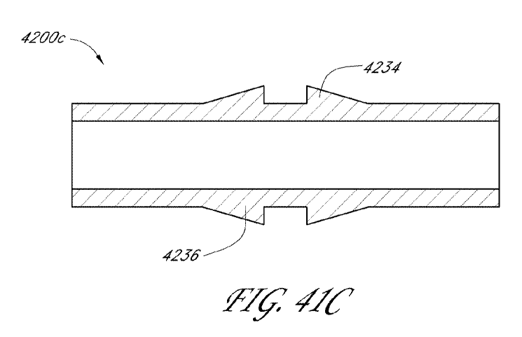

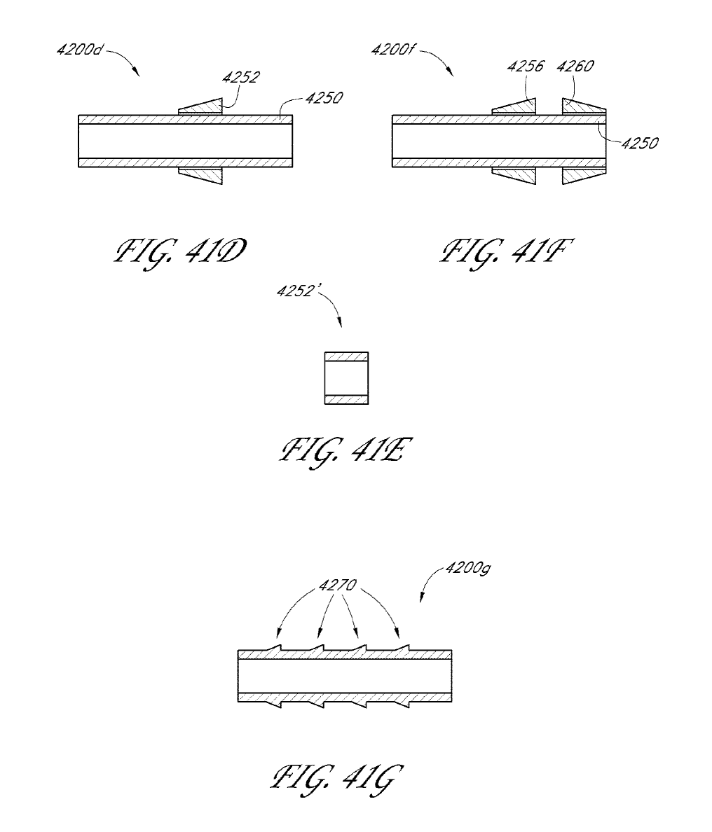

Embodiments Illustrated in FIG. 41A

FIGS. 41A-H show other embodiments of a system that can be used to perform a variety of methods or procedures. The shunts illustrated in FIGS. 41A-H include a feature(s) 4200 to engage the fibrous muscle adhesion 4210 that attaches the choroid to the sclera at its furthest anterior extent of the choroid. The feature 4200 can help prevent the stent from moving once implanted. The feature 4200 can also give the surgeon tactile feedback as to the ideal axial positioning of the device. Such a feature 4200 can be in the form of a circumferential groove, a protruding anchor, a flange, etc.

The shunt illustrated in FIG. 41B includes a feature 4200 formed by wire which is placed through a hole 4230. Preferably, the wire is preformed and elastic, which allows it to fold down during implantation using a delivery device 4240 as depicted.

In some embodiments, the retention feature(s) may be cut deep enough to make the body of a shunt made from plastic or metal flexible enough for insertion through bent tube, such as a cannula, and to conform to anatomy after placement. FIG. 41H shows a plurality of retention features on the body of a shunt which have been sized to weaken the wall of the shunt and to provide flexibility of the tube.

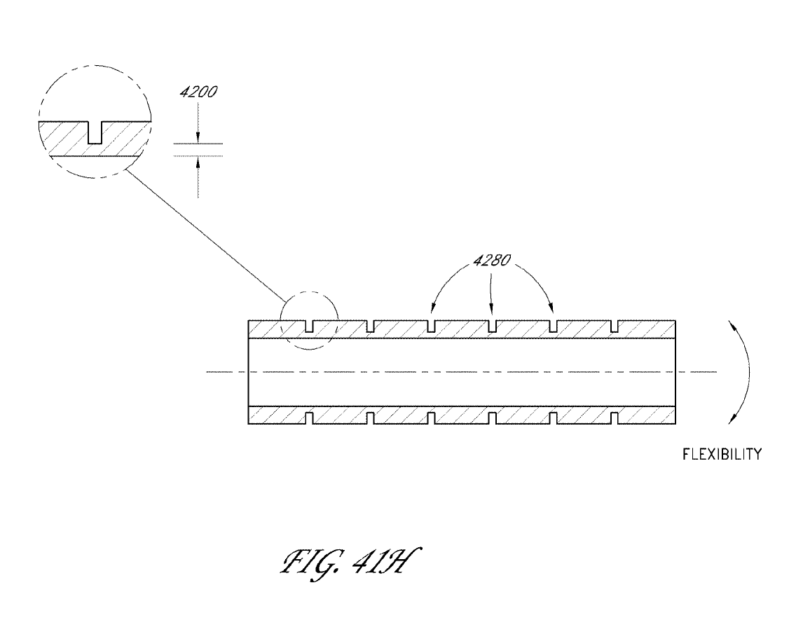

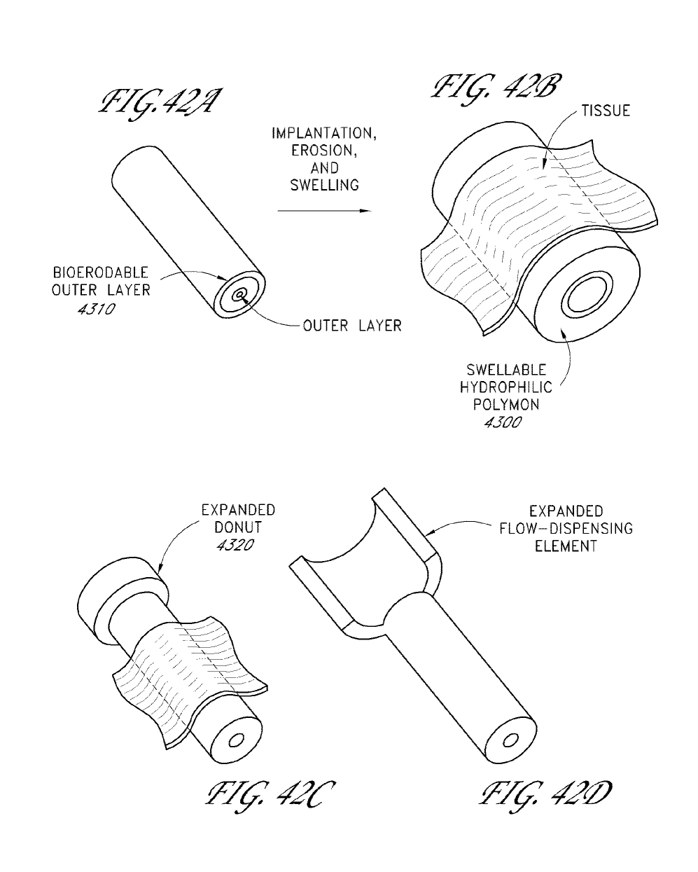

Embodiments Illustrated in FIGS. 42A-D

FIGS. 42A-D show other embodiments of a system that can be used to perform a variety of methods or procedures. The shunts illustrated in FIGS. 42A-D are made of a swellable hydrophilic polymer 4300. The swellable hydrophilic polymer can be, for example, swellable hydrophilic aliphatic polyurethane. Swelling of the polymer after insertion of the shunt can create a tight fit in the tissue, as shown in FIGS. 42A and 42B. The swellable material can be applied by, for example, dip coating, spray coating, or coextrusion to a core tubular structure comprised of a nonswellable polymeric or metal or ceramic material. Alternatively, the stent can be molded or extruded from the swellable hydrophilic material. In either of these cases, the outer surface can be covered by a thin layer of a biodegradable polymer 4310 such as polylactic acid, as shown in FIG. 42A. The layer of biodegradable polymer can prevent the swellable polymer from swelling until after it is implanted. A layer of viscoelastic may also accomplish this purpose. The swellable material may be formed as one or two “donuts” 4320 to further enhance retention of the stent, and prevention of anterior or posterior migration. The swellable material may also be designed to form a flow-dispersing component upon swelling.

### Variations

The shunt need not have a unitary configuration; that is, be formed of the same piece of material. For example, a proximal portion of the shunt can be formed of glass drawn to have at least one small diameter lumen. A distal portion of the shunt can be a cap formed of a different material.

If desired, more than one shunt of the same or different type may be implanted. For example, the shunts disclosed herein may be used in combination with trabecular bypass shunts, such as those disclosed in U.S. Patent Publication 2004/0050392 (Appendix A), and those described in U.S. Patent Publication 2005/0271704, filed Mar. 18, 2005, which is attached hereto as Appendix B and the entirety of which is incorporated herein by reference and made a part of this specification and disclosure. Additionally, implantation may be performed in combination with other surgical procedures, such as cataract surgery. All or a portion of the shunt may be coated, e.g. with heparin, preferably in the flow path, to reduce blood thrombosis or tissue restenosis.

If desired, a multiplicity of shunts having different flow capacities and/or lumen sizes may be implanted. For example, a single “large” lumen stent can be implanted first, and subsequent, depending on the pressure response to the first stent, a second can be added with potentially smaller flow capacity in order to “fine tune” the desired TOP. For example, the TOP of a first patient can safely be brought down to approximately 12-18 mm Hg, and once the flow capacity of the first stent is matched with the TOP reduction, a calculation can be made as to what additional outflow is required to achieve target pressures of, for example, approximately 8-12 mmHg. An appropriately sized stent can be added to accomplish the target pressure. Both stents can be proactively added at the same time based on calculated outflow requirements. Alternatively, the stents can be added sequentially as described above based on the measured effect of the first stent.

While certain embodiments of the disclosure have been described, these embodiments have been presented by way of example only, and are not intended to limit the scope of the disclosure. Indeed, the novel methods, systems, and devices described herein may be embodied in a variety of other forms. For example, embodiments of one illustrated or described shunt can be combined with embodiments of another illustrated or described shunt. Moreover, the shunts described above can be utilized for other purposes. For example, the shunts can be used to drain fluid from the anterior chamber to other locations of the eye or outside the eye. Furthermore, various omissions, substitutions and changes in the form of the methods, systems, and devices described herein may be made without departing from the spirit of the disclosure.

1-50. (canceled)

51. A method for reducing intraocular pressure in an eye of a mammal, comprising:
introducing an ocular implant through the anterior chamber of the eye, the ocular implant comprising an elongate outer shell with proximal and distal ends and being shaped to define at least one fluid flow passageway;
introducing the implant into eye tissue such that the distal end is in fluid communication with a physiological outflow pathway for ocular fluid and the proximal end is in fluid communication with the anterior chamber of the eye; and
ejecting from the ocular implant and into the physiological outflow space an expandable material, wherein upon expansion, the expandable material expands in one or more dimensions to create a void in the physiological outflow space more posterior in the eye than the distal end of the implant,
wherein aqueous humor is conducted between the proximal and distal ends of the implant and into the physiological outflow space, thereby reducing intraocular pressure and
wherein the expandable material is bioerodible.

52. The method of claim 51, wherein the expandable material bioerodes within about 2 weeks to 2.

53. The method of claim 51, wherein the fluid flow passageway comprises one or more inflow portions positioned at or near the proximal end of the implant and at least one outflow portion positioned substantially along a distal portion of the elongate outer shell, with at least one outflow portion being at the distal end of the implant.

54. The method according to claim 51, wherein the proximal end is positioned within the anterior chamber of the eye.

55. The method according to claim 54, wherein the distal end is positioned within the physiological outflow space.

56. The method according to claim 55, wherein the physiological outflow space is the supraciliary space or the suprachoroidal space.

57. The method of claim 51, wherein the implant has a length of between about 15 and 25 mm.

58. The method according to claim 51, wherein the expandable material is positioned in the implant prior to introducing the implant through the anterior chamber.

59. The method according to claim 51, wherein the expandable material is passed through the implant after introducing the implant through the anterior chamber.

60. The method according to claim 51, wherein the expandable material is encapsulated in a bioerodible material.

61. The method according to claim 60, wherein the material encapsulating the expandable material bioerodes within about 3 seconds to about 30 minutes.

62. A method for reducing intraocular pressure in an eye of a mammal, comprising:
introducing an ocular implant through the anterior chamber of the eye, the ocular implant comprising an elongate outer shell with proximal and distal ends and being shaped to define at least one fluid flow passageway;
introducing the implant into eye tissue such that the distal end is in fluid communication with a physiological outflow pathway for ocular fluid and the proximal end is in fluid communication with the anterior chamber of the eye; and
delivering into the physiological outflow space an expandable material by passing the expandable material alongside the outer shell of the implant, wherein upon expansion, the expandable material expands in one or more dimensions to create a void in the physiological outflow space,
wherein aqueous humor is conducted between the proximal and distal ends of the implant and into the physiological outflow space, thereby reducing intraocular pressure, and
wherein the expandable material is encapsulated in a bioerodible material.

63. The method of claim 62, wherein the material encapsulating the expandable material bioerodes within about 3 seconds to about 30 minutes.

64. The method of claim 62, wherein the fluid flow passageway comprises one or more inflow portions positioned at or near the proximal end of the implant and at least one outflow portion positioned substantially along a distal portion of the elongate outer shell, with at least one outflow portion being at the distal end of the implant.

65. The method according to claim 62, wherein the proximal end is positioned within the anterior chamber of the eye.

66. The method according to claim 65, wherein the distal end is positioned within the physiological outflow space.

67. The method according to claim 66, wherein the physiological outflow space is the supraciliary space or the suprachoroidal space.

68. The method of claim 62, wherein the expandable material is bioerodible, and wherein the expandable material bioerodes within about 2 weeks to 2 years.

69. The method according to claim 62, wherein the expandable material is positioned in the implant prior to introducing the implant through the anterior chamber.

70. The method according to claim 62, wherein the expandable material is passed through the implant after introducing the implant through the anterior chamber.

71. (canceled)

72. (canceled)

73. (canceled)
